(19) United States
(12) Patent Application Publication
(10) Pub. No.: US 2020/0297514 A1
(43) Pub. Date: Sep. 24, 2020

(54) MODULAR, ADJUSTABLE, ABOVE-KNEE PROSTHETIC SOCKET

(71) Applicant: OpenGait, Raleigh, NC (US)

(72) Inventors: Dustin Ryan Prescott, Raleigh, NC (US); Lindsay Katherine Sullivan, Raleigh, NC (US); Aaron John Fleming, Raleigh, NC (US)

(73) Assignee: OpenGait, Raleigh, NC (US)

(21) Appl. No.: 16/899,052

(22) Filed: Jun. 11, 2020

Related U.S. Application Data
(63) Continuation of application No. PCT/US2019/063634, filed on Nov. 27, 2019.
(60) Provisional application No. 62/889,795, filed on Aug. 21, 2019.
(60) Provisional application No. 62/773,323, filed on Nov. 30, 2018.
(60) Provisional application No. 62/889,795, filed on Aug. 21, 2019.

(51) Int. Cl. A61F 2/80

(52) U.S. Cl. CPC A61F 2/80; A61F 2002/785

(57) ABSTRACT

Disclosed is a modular prosthetic socket with highly adjustable components for fit. The socket, in one embodiment, includes at least one paddle and at least one rod, wherein straps secure the socket to a residual limb. The at least one paddle and at least one rod are adjustable by way of a baseplate assembly, which allow for radial and circumferential adjustability of the attached components.

### CROSS REFERENCES TO RELATED APPLICATIONS

This application is related to and claims priority from the following US patents and patent applications. This application claims priority to and the benefit of U.S. Provisional Patent Application No. 62/889,795, filed Aug. 21, 2019. This application is also a continuation of Patent Cooperation Treaty Application No. PCT/US2019/063634, filed Nov. 27, 2019, which claims priority to and the benefit of U.S. Provisional Patent Application No. 62/773,323, filed Nov. 30, 2018, and U.S. Provisional Patent Application No. 62/889,795, filed Aug. 21, 2019. Each of these applications is incorporated herein by reference in its entirety.

### BACKGROUND OF THE INVENTION

### 1. Field of the Invention

The presently disclosed subject matter relates to apparatuses and methods for artificial limbs. In particular, the subject matter relates to above knee prosthetic legs.

### 2. Description of the Prior Art

Due to the large variations in residual limb shapes and sizes, sockets for prosthetics are often handmade by a highly-trained prosthetists for those with residual limbs, which is a labor-intensive process. For example, one socket can sometimes take up to nine hours to produce. This process further requires extensive equipment and costly materials.

Further, these custom solutions often require a specialist trained in fitting the limb once it is made. Additionally, conventional sockets often require a manufacturer to configure and adjust prosthetic connections to a knee of the sockets. Moreover, users often experience major volume changes in a residual limb both throughout the course of a day as well as over the course of the year, which can prevent usage of many custom prostheses. While some products offer a level of adjustability, these sockets usually require castings, measurements, or even three-dimensional digital profiles to be created and used by a specialist to fit the socket to the user.

There can be even more challenges for individuals in need of prosthetics, including for those in developing countries. For example, obtaining measurements and fabricating custom solutions for a prosthetic limb can often be a barrier to those without access to resources and medical professionals. Furthermore, amputation techniques differ from country to country. Methods by which the muscles are terminated often affects shapes and needs of a residual limb and therefore also changes requirements for a prosthesis.

Hence, there is a need for lower limb prosthetics that are less expensive, more easily customizable, and which can accommodate a range of medical methods and access.

Prior art patent documents include the following:

U.S. Pat. No. 10,251,710 for Method and system for assembly of a modular prosthetic socket based on residual limb metrics by inventor Hurley, et. al, filed May 17, 2018 and issued Apr. 9, 2019, is directed to a method of providing a modular prosthetic socket for a residual limb of a patient may involve receiving digital data defining a three-dimensional digital profile of the residual limb and selecting prosthetic socket components from component-specific inventories, based at least in part on the digital profile. The selected prosthetic socket components may include: multiple longitudinal struts; one or more proximal brim members for attachment to the longitudinal struts; and a distal socket base to which the longitudinal struts attach at or near their distal ends. The method may further involve providing the selected prosthetic components to an operator for assembling into the modular prosthetic socket. The prosthetic socket, when assembled, defines an internal space substantially complementary to the profile of the residual limb.

U.S. Pat. No. 10,369,027 for Adaptable socket system, method, and kit by inventor Alley, filed Feb. 2, 2012 and issued Aug. 8, 2019, is directed to an adjustable socket system, method, and kit for use with prosthetic devices or orthotic, orthopedic, or exoskeletal support devices that includes a paddle and a compressing device coupled to the paddle. The paddle is chosen from a plurality of paddles of different shapes in such a way that the paddle inner surface is substantially coextensive with a soft tissue area overlying skeletal structures. The compressing device presses the paddle against the soft tissue area in order to minimize the motion of the paddle relative to the underlying skeletal structures without causing discomfort to the user or compressing areas not in contact with the paddle, allowing compressed tissue to flow into uncompressed areas. The system, method, and kit can include an external tool, sensors, actuators, and controllers, to assist fitting and for adjustment after fitting. A stabilizer can be added to resist the bending force on the paddle.

U.S. Pat. No. 10,172,728 for Transtibial prosthetic socket with textile jacket by inventor Hurley, et. al, filed Nov. 23, 2016 and issued Jan. 8, 2019, is directed to a transtibial prosthetic socket frame may include a distal base assembly having a base plate, a carriage configured to support a socket suspension arrangement, and a distal prosthetic component connector. The distal base assembly supports a set of struts that includes two anterior struts and a single posterior strut. The set of struts and distal base assembly collectively define a prosthetic socket cavity having a central longitudinal axis and a residual limb hosting volume. The distal prosthetic component connector has a connecting adapter that is rotatable with respect to the prosthetic socket, and moveable with respect to the base plate between being aligned with the prosthetic socket's central longitudinal axis and a position offset therefrom.

US Publication No. 2016/0045340 for radial volume adjustment device by inventor Vaughan, et. al, filed Mar. 27, 2014 and published Feb. 18, 2016, is directed to a radial volume adjustment device. The device includes a connection plate. The device further includes a socket wall comprising a plurality of socket wall components, at least one of the plurality of socket wall components comprising a channel, the channel forming an arcuate path curving towards a center of the connection plate. The device further includes at least one attachment member configured to couple with the connection plate and pass through the at least one channel such that the at least one socket wall component may move in a path defined by the at least one attachment member and the channel. The movement of the at least one attachment member causes a volume defined by the socket wall to change.

US Publication No. 2018/0296373 for Adjustable socket system by inventor Granz, filed Jun. 26, 2018 and published Oct. 18, 2018, is directed to a prosthetic connector system for use with a prosthetic socket having a distal end includes a plate defining a plurality of holes and attachable to the distal end of the prosthetic socket. A component is removably attachable to the plate via the plurality of holes and arranged to connect a prosthesis to the prosthetic socket. The plate defines an outer periphery having an asymmetrical configuration contoured to substantially correspond to at least a portion of the component when the component is attached to the plate via at least some of the holes.

US Publication No. 2018/0235785 for Adjustable socket system by inventor Bache, et. al, filed Apr. 18, 2018 and published Aug. 23, 2018, is directed to an adjustable socket system includes a distal portion and proximal portion. An axis extends between the distal and proximal portions. A plurality of struts are connected to the distal portion and distributed circumferentially about the axis. The struts at least in part define a receiving volume adapted to receive a residual limb and are movable between an expanded configuration in which at least some of the struts are moved radially outward relative to the axis to loosen the fit of the adjustable system, and a closed configuration in which at least some of the struts are moved radially inward relative to the expanded configuration to tighten the fit of the adjustable socket system. A tightening system is operatively connected to the struts and arranged to differentially tighten and loosen the fit of the adjustable socket system on one or more areas of the residual limb via a single input.

US Publication No. 2019/0060089 for Transfemoral level interface system using compliant members by inventor Martin, filed Aug. 30, 2017 and published Feb. 28, 2019, is directed to a transfemoral prosthetic level socket system for a user's lower limb comprising modular socket components fitted to the individual user's residual limb having a mounting point for an attachment, at least one compliant member attached to at least one stabilizing unit, and at least one second compliant member attached to at least one stabilizing unit wherein the first compliant member and the second compliant member work in cooperation with the stabilizing unit(s) to control bone position and support the limb within the interface.

### SUMMARY OF THE INVENTION

The present invention relates to apparatuses and methods for artificial limbs. In one embodiment, the invention is directed to above knee prosthetic legs. However, this invention is further operable to be applied in use for below the knee prosthetic limbs, above the elbow prosthetic arms, and below the elbow prosthetic arms.

It is an object of this invention to employ specific components to provide a prosthetic limb that is highly customizable and does not require complex measurements and casting to provide a secure and usable fit. This and other objects are achieved in whole or in part by the present invention. A modular design ensures the device is highly customizable based on readily available parts to accommodate for the variability in residual limb shapes and sizes.

In one embodiment, the present invention includes a socket for a prosthetic leg, comprising: at least three paddles, including a femoral paddle, a hip paddle and an ischial seat paddle; a baseplate assembly; and at least one front strap and at least one rear strap; wherein the at least one front strap and the at least one rear strap are attached to the hip paddle and the ischial seat paddle; wherein the femoral paddle is attached to the at least one front strap; wherein the baseplate assembly is connected to the hip paddle via at least one rod, and wherein the baseplate assembly is connected to the ischial seat paddle via at least two rods; wherein the baseplate assembly includes an ischial seat connector, a hip rod connector, and a knee connector; wherein the hip rod connector is adjustable in at least one first radial direction and at least one first circumferential direction; wherein the hip rod connector is angled between 5 degrees and 10 degrees from a top surface of the baseplate assembly; wherein the ischial seat connector is continuously, circumferentially adjustable; and wherein the knee connector is adjustable in at least one second radial direction and at least one second circumferential direction. In some embodiments, the socket further comprises a femoral paddle, wherein the femoral paddle is attached to the at least one front strap.

In another embodiment, the present invention includes a socket for a prosthetic leg, comprising: at least two paddles, including a hip paddle and an ischial seat paddle; a baseplate assembly; and at least one front strap and at least one rear strap; wherein the at least one front strap and the at least one rear strap are attached to the hip paddle and the ischial seat paddle; wherein the hip paddle and the ischial seat paddle are each connected to the baseplate assembly via at least one rod; wherein the baseplate assembly includes an ischial seat connector and a hip rod connector; wherein the hip rod connector is adjustable in at least one radial direction and at least one circumferential direction; and wherein a hip paddle attachment point of a first strap of the at least one front strap is located a greater distance from the baseplate assembly than an ischial seat paddle attachment point of the first strap of the at least one front strap, such that the first strap of the at least one front strap is configured to be approximately parallel to an inguinal ligament.

In yet another embodiment, the present invention includes a socket for a prosthetic leg, comprising: at least three paddles, including a femoral paddle, a hip paddle and an ischial seat paddle, a baseplate assembly, and at least one front strap and at least one rear strap, wherein the femoral paddle is attached to the at least one front strap, wherein the at least one front strap and the at least one rear strap are attached to the hip paddle and the ischial seat paddle, wherein the hip paddle and the ischial seat paddle are each connected to the baseplate assembly via at least one rod, wherein the baseplate assembly includes an ischial seat connector and a hip rod connector, wherein the baseplate assembly includes a top surface and a bottom surface, wherein the ischial seat connector and the hip rod connector are attached to the top surface of the baseplate assembly, wherein the hip rod connector is angled between 5 degrees and 10 degrees from a top surface of the baseplate assembly, and wherein the hip rod connector is adjustable in at least one radial direction and at least one circumferential direction.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1A illustrates a front view of a lower half of a human body, illustrating regions of support for attachment of a prosthesis.

FIG. 1B illustrates a rear view of a lower half of a human body, illustrating regions of support for attachment of a prosthesis.

FIG. 2 illustrates a front isometric view of a socket, illustrating the three main paddles according to one embodiment of the present invention.

FIG. 3 illustrates a side view of a socket, illustrating three main paddles according to one embodiment of the present invention.

FIG. 4 illustrates a top view of a socket, illustrating a position of the three main paddles according to one embodiment of the present invention.

FIG. 5A illustrates a top perspective rear view of a baseplate, illustrating mounting blocks in a posterior position according to one embodiment of the present invention.

FIG. 5B illustrates a top perspective rear view of a baseplate, illustrating mounting blocks in an anterior position according to one embodiment of the present invention.

FIG. 5C illustrates a bottom perspective bottom view of a baseplate, including an adjustable location of a lower limb pyramid connector according to one embodiment of the present invention.

FIG. 6A illustrates a top view of a mounting block according to one embodiment of the present invention.

FIG. 6B illustrates an isometric view of a mounting block according to one embodiment of the present invention.

FIG. 6C illustrates a perspective section view of a mounting block with an angled receptacle, according to one embodiment of the present invention.

FIG. 6D illustrates a side section view of a mounting block with an angled receptacle, according to one embodiment of the present invention.

FIG. 7A illustrates a top perspective view of a socket, illustrating three main paddles and straps, according to one embodiment of the present invention.

FIG. 7B illustrates a front perspective view of a socket, illustrating three main paddles and three front straps, according to one embodiment of the present invention.

FIG. 7C illustrates a rear view of a socket, including a rear pad attached to circumferential straps, according to one embodiment of the present invention.

FIG. 7D illustrates a front view of a socket securing a residual limb, including paddle placement, according to one embodiment of the present invention.

FIG. 7E illustrates a first perspective view of a socket with a hip paddle and an ischial seat paddle according to one embodiment of the present invention.

FIG. 7F illustrates a second perspective view of a socket with a hip paddle and an ischial seat paddle according to one embodiment of the present invention.

FIG. 7G illustrates a second perspective view of a socket with a hip paddle and an ischial seat paddle according to one embodiment of the present invention.

FIG. 7H illustrates a rear view of a socket securing a residual limb, including paddle placement, according to one embodiment of the present invention.

FIG. 7I illustrates a first perspective view of a socket with a reversible hip paddle and a reversible ischial seat paddle according to one embodiment of the present invention.

FIG. 7J illustrates a second perspective view of a socket with a reversible hip paddle and a reversible ischial seat paddle according to one embodiment of the present invention.

FIG. 7K illustrates a third perspective view of a socket with a reversible hip paddle and a reversible ischial seat paddle according to one embodiment of the present invention.

FIG. 8A illustrates a perspective view of a hip paddle, including a hip rod, according to one embodiment of the present invention.

FIG. 8B illustrates a front view of a hip paddle, including a hip rod, according to one embodiment of the present invention.

FIG. 8C illustrates a front exploded view of a hip paddle, including a backplate and a hip plate, according to one embodiment of the present invention.

FIG. 8D illustrates a rear exploded view of a hip paddle, including a backplate and a hip plate, according to one embodiment of the present invention.

FIG. 8E illustrates a rear perspective view of a hip paddle backplate with symmetric holes, according to one embodiment of the present invention.

FIG. 8F illustrates a rear perspective view of a hip paddle hip plate with symmetric holes, according to one embodiment of the present invention.

FIG. 8G illustrates a front perspective view of a hip paddle backplate with symmetric holes, according to one embodiment of the present invention.

FIG. 8H illustrates a front perspective view of a hip paddle hip plate with symmetric holes, according to one embodiment of the present invention.

FIG. 9A illustrates a rear view of an ischial seat paddle, including backplates, according to one embodiment of the present invention.

FIG. 9B illustrates a perspective view of an ischial seat paddle, including support rods, according to one embodiment of the present invention.

FIG. 9C illustrates a rear view of an ischial seat paddle, including an ischial plate, according to one embodiment of the present invention.

FIG. 9D illustrates a front perspective view of an ischial seat paddle with higher strap positioning according to one embodiment of the present invention.

FIG. 9E illustrates a rear perspective view of an ischial seat paddle with higher strap positioning according to one embodiment of the present invention.

FIG. 9F illustrates a front exploded view of an ischial seat paddle with higher strap positioning according to one embodiment of the present invention.

FIG. 9G illustrates a rear exploded view of an ischial seat paddle with higher strap positioning according to one embodiment of the present invention.

FIG. 10A illustrates a perspective view of an ischial backplate with through-holes, according to one embodiment of the present invention.

FIG. 10B illustrates a rear perspective view of an ischial backplate with rod channels and strap channels, according to one embodiment of the present invention.

FIG. 11 illustrates a perspective view of a femoral paddle, illustrating contours similar to that of quadriceps muscles, according to one embodiment of the present invention.

FIG. 12 illustrates an isometric view of a baseplate assembly, including a fully extended hip rod connector, according to one embodiment of the present invention.

FIG. 13A illustrates a perspective view of a hip rod connector including an extruded foot, according to one embodiment of the present invention.

FIG. 13B illustrates a front view of a hip rod connector, illustrating an angled cylinder, according to one embodiment of the present invention.

FIG. 14A illustrates a bottom view of a baseplate, including ischial seat connector bores and a center bore, according to one embodiment of the present invention.

FIG. 14B illustrates a perspective view of a baseplate, knee-baseplate slider, and a connector pin, according to one embodiment of the present invention.

FIG. 14C illustrates a top view of a baseplate, including ischial seat connector bores and a center bore, according to one embodiment of the present invention.

FIG. 14D illustrates a bottom view of a baseplate, including ischial seat connector bores and a center bore, according to one embodiment of the present invention.

FIG. 15A illustrates a bottom view of a baseplate and knee connector according to one embodiment of the present invention.

FIG. 15B illustrates a top perspective view of a knee connector, illustrating connection points for connection to a prosthetic limb, according to one embodiment of the present invention.

FIG. 16A illustrates a top perspective view of an ischial seat connector, including ischial seat bores, according to one embodiment of the present invention.

FIG. 16B illustrates a side perspective view of an ischial seat connector, including securing bores, according to one embodiment of the present invention.

FIG. 16C illustrates an exploded view of an ischial seat connector and a baseplate, illustrating positioning of an ischial seat connector over ischial seat connector bores, according to one embodiment of the present invention.

FIG. 16D illustrates a section view of an ischial seat connector at a plane bisecting an ischial seat bore, illustrating an angle of the ischial seat bore, according to one embodiment of the present invention.

FIG. 17A illustrates an anterior view of a femoral paddle, including securement plates and front straps, according to one embodiment of the present invention.

FIG. 17B illustrates a top view of a femoral paddle, illustrating one level of curvature of a front surface and front straps, according to one embodiment of the present invention.

FIG. 17C illustrates a top view of a femoral paddle, illustrating a second level of curvature of a front surface and front straps, according to one embodiment of the present invention.

FIG. 17D illustrates a top view of a femoral paddle, illustrating a third level of curvature of a front surface and front straps, according to one embodiment of the present invention.

FIG. 18A illustrates a rear view of a rear pad, including pad sleeves and rear straps, according to one embodiment of the present invention.

FIG. 18B illustrates a top view of a rear pad, illustrating a pad with no curvature, according to one embodiment of the present invention.

FIG. 18C illustrates a top view of a rear pad, illustrating a pad with minor curvature, according to one embodiment of the present invention.

FIG. 19A illustrates a side view of padding for a hip paddle according to one embodiment of the present invention.

FIG. 19B illustrates a side view of padding for an ischial seat paddle according to one embodiment of the present invention.

FIG. 19C illustrates a side view of padding for a femoral paddle according to one embodiment of the present invention.

FIG. 20A illustrates a side view of a hip paddle and hip paddle padding, illustrating complementary shapes, according to one embodiment of the present invention.

FIG. 20B illustrates a side view of an ischial seat paddle and ischial seat paddle padding, illustrating complementary shapes, according to one embodiment of the present invention.

FIG. 20C illustrates a side view of a femoral paddle and femoral paddle padding, illustrating complementary shapes, according to one embodiment of the present invention.

FIG. 21A illustrates a front view of a tissue containment unit and a vertical zone of frictional material, according to one embodiment of the present invention.

FIG. 21B illustrates a rear view of a tissue containment unit and vertical zones of frictional material, according to one embodiment of the present invention.

FIG. 21C illustrates a medial side view of a tissue containment unit and vertical zones of frictional material, according to one embodiment of the present invention.

FIG. 21D illustrates a lateral side view of a tissue containment unit and vertical zones of frictional material, according to one embodiment of the present invention.

FIG. 21E illustrates a front view of a tissue containment unit and targeted zones of frictional material, according to one embodiment of the present invention.

FIG. 21F illustrates a rear view of a tissue containment unit and targeted zones of frictional material, according to one embodiment of the present invention.

FIG. 21G illustrates a medial side view of a tissue containment unit and targeted zones of frictional material, according to one embodiment of the present invention.

FIG. 21H illustrates a lateral side view of a tissue containment unit and targeted zones of frictional material, according to one embodiment of the present invention.

FIG. 21I illustrates a front view of a tissue containment unit and horizontal zones of frictional material, according to one embodiment of the present invention.

FIG. 21J illustrates a rear view of a tissue containment unit and horizontal zones of frictional material, according to one embodiment of the present invention.

FIG. 21K illustrates a medial side view of a tissue containment unit and horizontal zones of frictional material, according to one embodiment of the present invention.

FIG. 21L illustrates a lateral side view of a tissue containment unit and horizontal zones of frictional material, according to one embodiment of the present invention.

FIG. 22A illustrates a front view of a X-shaped tissue containment unit and reinforcement element, according to one embodiment of the present invention.

FIG. 22B illustrates a rear view of a X-shaped tissue containment unit and reinforcement element, according to one embodiment of the present invention.

FIG. 22C illustrates a bottom view of a X-shaped tissue containment unit and reinforcement element, according to one embodiment of the present invention.

FIG. 22D illustrates a front view of a Y-shaped tissue containment unit and reinforcement element, according to one embodiment of the present invention.

FIG. 22E illustrates a rear view of a Y-shaped tissue containment unit and reinforcement element, according to one embodiment of the present invention.

FIG. 22F illustrates a bottom view of a Y-shaped tissue containment unit and reinforcement element, according to one embodiment of the present invention.

### DETAILED DESCRIPTION

The present invention is generally directed to a socket for a prosthetic leg. The present invention provides an increased level of adjustability and a low-cost alternative to highly customized, non-adjustable sockets. A set of supporting paddles in combination with an adjustable baseplate and straps allows for a prosthetic socket to be fit to a residual limb of a user without the need for a custom fabrication. Additionally, the adjustable nature of the prosthetic socket allows for several degrees of freedom to ensure comfort and support as limb size changes throughout usage.

In one embodiment, the present invention includes a socket for a prosthetic leg, comprising: at least three paddles, including a femoral paddle, a hip paddle and an ischial seat paddle; a baseplate assembly; and at least one front strap and at least one rear strap; wherein the at least one front strap and the at least one rear strap are attached to the hip paddle and the ischial seat paddle; wherein the femoral paddle is attached to the at least one front strap; wherein the baseplate assembly is connected to the hip paddle via at least one rod, and wherein the baseplate assembly is connected to the ischial seat paddle via at least two rods; wherein the baseplate assembly includes an ischial seat connector, a hip rod connector, and a knee connector; wherein the hip rod connector is adjustable in at least one first radial direction and at least one first circumferential direction; wherein the hip rod connector is angled between 5 degrees and 10 degrees from a top surface of the baseplate assembly; wherein the ischial seat connector is continuously, circumferentially adjustable; and wherein the knee connector is adjustable in at least one second radial direction and at least one second circumferential direction. In some embodiments, the socket further comprises a femoral paddle, wherein the femoral paddle is attached to the at least one front strap.

In another embodiment, the present invention includes a socket for a prosthetic leg, comprising: at least two paddles, including a hip paddle and an ischial seat paddle; a baseplate assembly; and at least one front strap and at least one rear strap; wherein the at least one front strap and the at least one rear strap are attached to the hip paddle and the ischial seat paddle; wherein the hip paddle and the ischial seat paddle are each connected to the baseplate assembly via at least one rod; wherein the baseplate assembly includes an ischial seat connector and a hip rod connector; wherein the hip rod connector is adjustable in at least one radial direction and at least one circumferential direction; and wherein a hip paddle attachment point of a first strap of the at least one front strap is located a greater distance from the baseplate assembly than an ischial seat paddle attachment point of the first strap of the at least one front strap, such that the first strap of the at least one front strap is configured to be approximately parallel to an inguinal ligament.

In yet another embodiment, the present invention includes a socket for a prosthetic leg, comprising: at least three paddles, including a femoral paddle, a hip paddle and an ischial seat paddle, a baseplate assembly, and at least one front strap and at least one rear strap, wherein the femoral paddle is attached to the at least one front strap, wherein the at least one front strap and the at least one rear strap are attached to the hip paddle and the ischial seat paddle, wherein the hip paddle and the ischial seat paddle are each connected to the baseplate assembly via at least one rod, wherein the baseplate assembly includes an ischial seat connector and a hip rod connector, wherein the baseplate assembly includes a top surface and a bottom surface, wherein the ischial seat connector and the hip rod connector are attached to the top surface of the baseplate assembly, wherein the hip rod connector is angled between 5 degrees and 10 degrees from a top surface of the baseplate assembly, and wherein the hip rod connector is adjustable in at least one radial direction and at least one circumferential direction.

None of the prior art discloses a prosthetic socket with highly customizable components, including the combination of hip and ischial seat paddles with a baseplate assembly, wherein the baseplate assembly includes adjustable elements for modifying and securing positions for the paddles.

Compared to traditional sockets, which function to cover and put pressure on nearly all of a residual limb, this adjustable socket applies pressure to a residual limb in anatomically relevant locations where high levels of pressure can be safely applied. To ensure that the pressure is consistently applied in these locations regardless of limb size, the socket is, in one embodiment, a suspended-type socket assembly that adjustable radially, circumferentially, and vertically. The assembly addresses the extensive time, cost, and customization issues that prosthetists face and allows users to make modifications to the system based on daily fluctuations in residual limb dimensions. The system eliminates the casting process previously required in prosthetic construction and provides a ready to use, off-the-shelf socket that is operable to be adjusted to fit a user in very little time using few widely-available tools, resulting in a socket with appropriate fit at a much lower cost than traditional methods.

The prosthetic socket disclosed herein further provides a rugged alternative to complex or expensive prostheses, which a user may not wish to expose to a particular environment (e.g., during airline travel, near or in water or sand, at times requiring quick or repeated removal, or times of increased other risk of damage or theft).

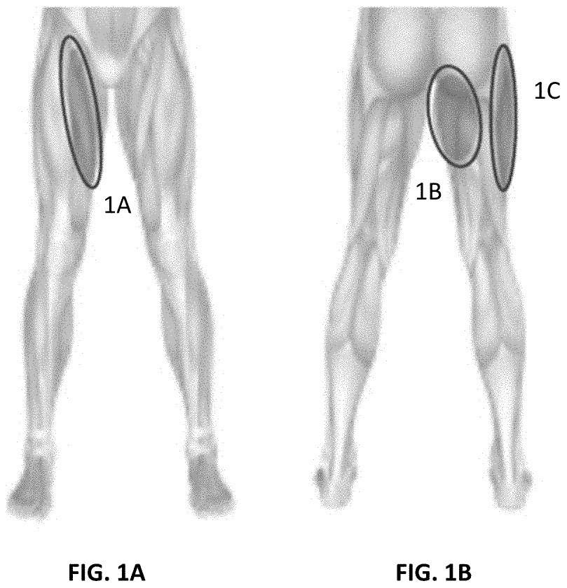

Several body locations within typical transfemoral sockets have been identified as optimal for applying pressure to secure the socket to an amputated limb. In on embodiment, these areas are illustrated in FIG. 1, which provide comfortable and supportive locations for attachment and reduce the potential for negative effects such as pinching, bruising, sores, etc. The locations are illustrated are: a femoral triangle region (1A), an ischial tuberosity or ischial seat (1B), and an outer hip/thigh region (1C). The femoral triangle 1A follows the femur, which in one embodiment, is uncontained due to amputation or a congenital condition. The ischial seat 1B is the location that supports a majority of weight when individuals sit, and is therefore a desirable location for supporting and translating force between the socket and the user. The outer hip region 1C provides medio-lateral (i.e., left to right) stability for the residual limb within the socket.

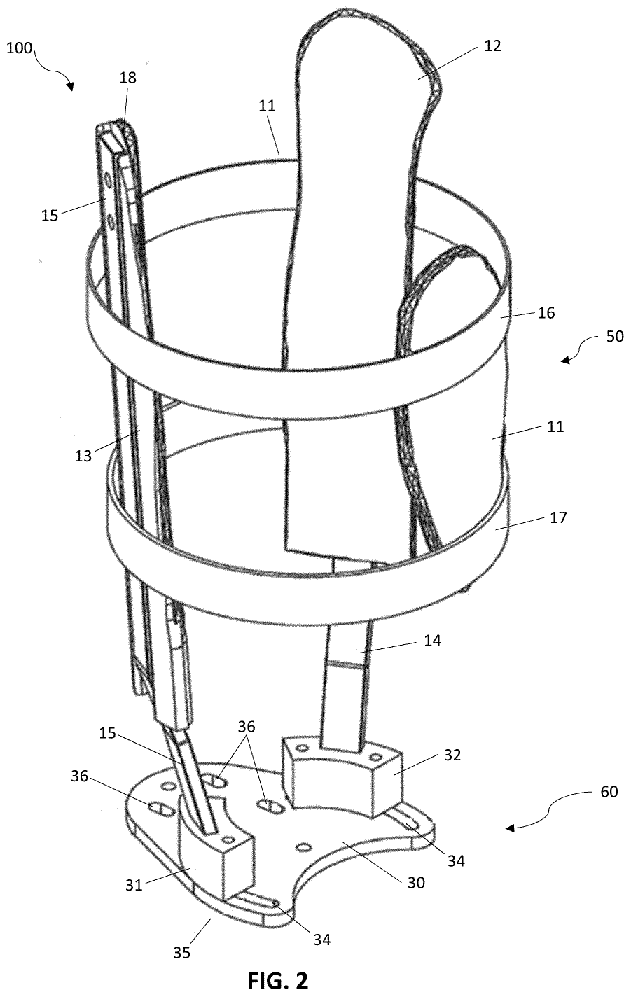

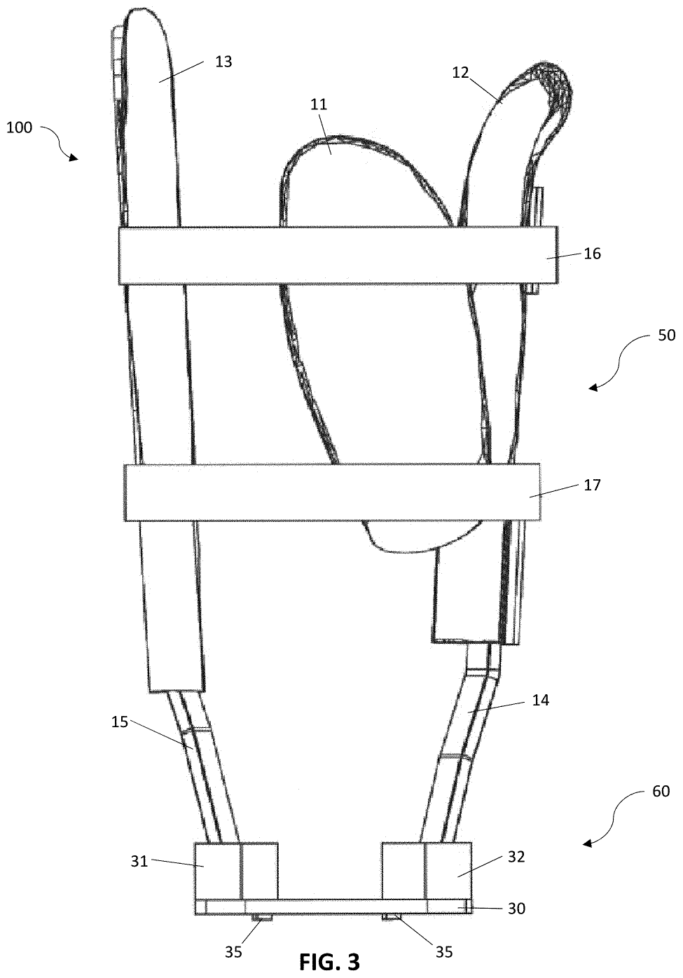

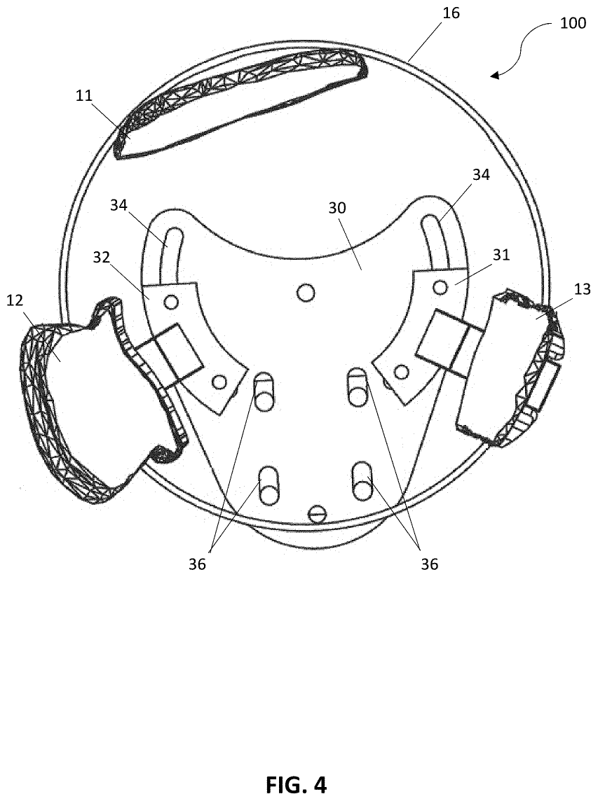

FIGS. 2, 3, and 4 depict an example socket assembly 100. FIG. 2 is an isometric view of socket assembly 100, FIG. 3 is a front view of socket assembly 100, and FIG. 4 is a top view of socket assembly 100. In the illustrated embodiment, the socket assembly is operable to attach to the limb using three paddles: a femoral paddle 11, an ischial seat paddle 12, and a hip paddle 13. Each paddle is designed with a general contour related to the respective pressure locations 1A, 1B, or 1C, respectively. Two adjustable vertical support bars, a medial strut 14 and a lateral strut 15, connect a proximal body attachment portion 50 to the distal baseplate assembly 30, which is located in a distal region 60. In the illustrated embodiment of FIG. 2, an ischial seat paddle 12 and a hip paddle 13 are adjustably mounted onto medial strut 14 and a lateral strut 15, respectively. Paddles 12 and 13 are operable to move up and down vertically, for example, via a slotted connection 18 to the struts. These adjustments allow for the socket to accommodate a range of heights and limb sizes. Medial strut 14 and lateral strut 15 extend from the proximal body attachment portion 50 to distal baseplate assembly 30.

FIGS. 2, 3, and 4, further illustrate circumferential straps 16 and 17, which are attached to struts 14 and 15. In the illustrated embodiment, straps 16 and 17 are fixedly attached to struts 14 and 15 in such a way that the ischial seat paddle 12 and hip paddle 13 are operable to be adjusted along the struts without interference. Straps 16 and 17, in further embodiments, include slidable mounting points (not shown) on struts 14 and 15. The paddles are further configured, in one embodiment, to fit between the straps and the user's body, which ensures protection of underlying tissue. Straps 16 and 17 are further operable to be positioned either horizontally or at an angle, in order to secure the socket above the quadriceps muscle and prevent the socket assembly from slipping.

The struts, straps, and paddles are further operable to be made of any suitable materials. The struts, in one embodiment, are constructed from a rigid material that has sufficient mechanical strength to support limb movement and load, while taking into consideration weight and safety, such as aluminum, steel, or carbon-fiber reinforced composites.

In another embodiment, the straps are constructed from a uniform material or a combination of materials. For example, in one embodiment, the straps are constructed from flexible materials that are compatible with skin contact, such as knit or non-woven fabric, breathable plastics, natural or synthetic rubber, foam, etc. Similarly, in one embodiment, the paddles are formed from one or more layers. For example, in one embodiment, at least one of the paddles includes a rigid portion for stability and strength and a cushioned portion for comfort.

A femoral paddle 11 is further operable to be attached to socket assembly 100 in a different manner than the ischial seat paddle 12 or the hip paddle 13. In the illustrated embodiment, the femoral paddle 11 is slidably attached to straps 16 and 17. This gives femoral paddle 11 considerable freedom of movement for placement over the femoral region 1A. The femoral paddle 11 is operable to move vertically, slide circumferentially (i.e., around perimeter of the leg), and/or rotate in a vertical plane. The femoral paddle 11, in one embodiment is constructed with a curvature that is convex with corresponding to an intended contact region of the body. The convex shape aids in correct ergonomic placement of the paddle to improve the pressure profile on the contacted tissue and to avoid restricting blood flow and other negative impacts.

To ensure that pressure is consistently applied to locations 1A, 1B, and 1C regardless of limb size, the proximal body attachment portion 50 of socket assembly 100 is adjustable both vertically, as described hereinabove, and also circumferentially. In the circumferential direction, the socket assembly 100 is operable to be tightened by straps 16 and 17. The straps comprise, in one embodiment, a strap that is releasably adjustable, such as but not limited to a ratcheting strap with a release mechanism, and the straps are constructed to be operated independently from one another. This retention method improves a speed of attachment and ease of use, and it also allows a user to modify a socket fit to accommodate dimensional fluctuations that are common in terminated limbs. For example, a volumetric area of a terminated limb often changes significantly on a daily, monthly, or yearly basis due to, for example, fluid retention. Some conventional socket assemblies cannot accommodate these dimensional swings or progressions, causing a user to delay wearing these limbs in the short term or requiring the user to acquire new artificial limbs over the long term. By providing circumferential adjustment in straps 16 and 17, to the socket promotes the use a single prosthetic limb for more hours a day or over a longer period of time. Moreover, the provided adjustments are easily and intuitively accessible by the user without assistance.

A further advantage of the straps is the potential elimination of the need for a liner element for many amputees. Liners are typically composed of a nonbreathable material, which can lead to perspiration and discomfort. Avoidance of a liner reduces a cost and complexity of the socket and improves user comfort and ease of hygiene.

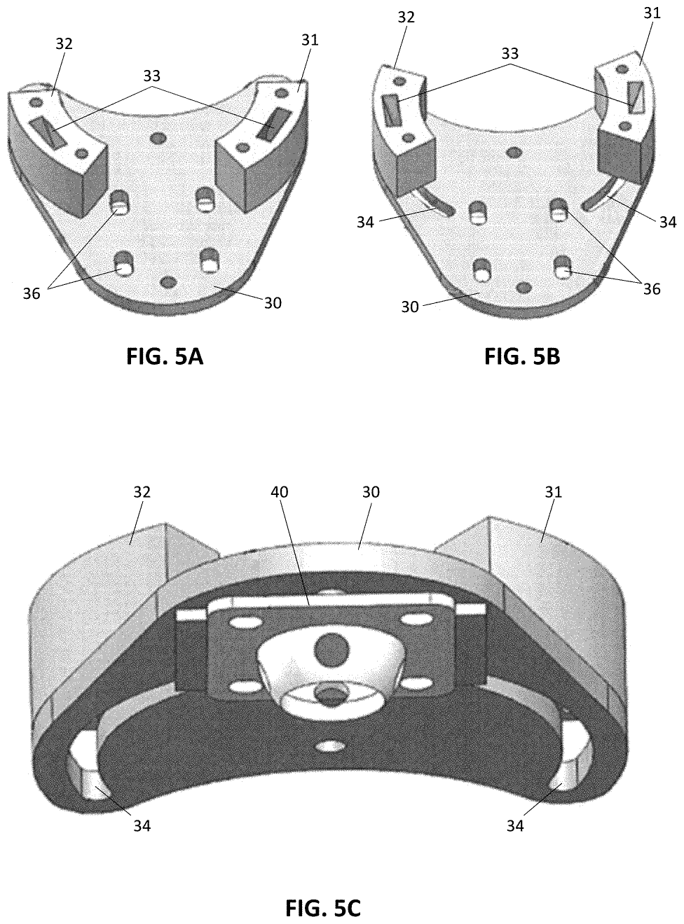

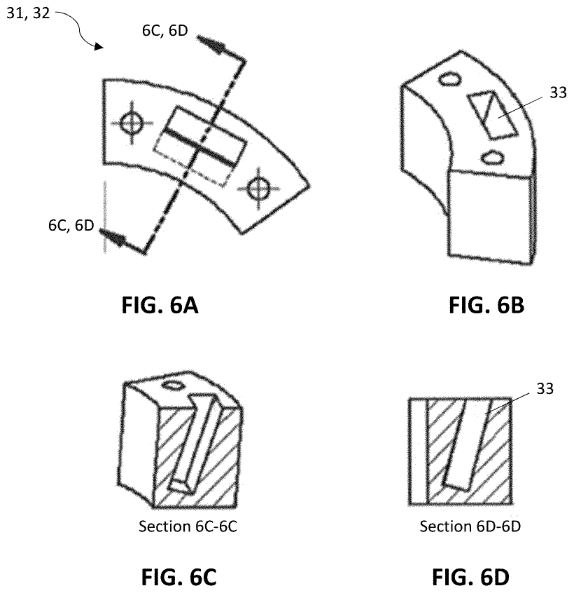

FIGS. 5A, 5B, and 5C illustrate a baseplate 30 in further detail. Baseplate 30 includes additional adjustment features. For example, baseplate 30 has slidable mounting blocks 31 and 32 that are used to secure struts 14 and 15. Mounting blocks 31 and 32 are mounted to a top or proximal side of baseplate 30 and have receiving features for attaching to struts 14 and 15. For example, as illustrated in FIG. 6, mounting blocks 31 and 32, in one embodiment, have angled receptacles 33 into which the struts are fitted. One or both of the struts 14 and 15 are secured in a receptacle 33 by one or more retention features, such as a set screw, pin, or clamp. FIG. 5A depicts mounting blocks 31 and 32 in a more rearward (i.e., posterior) position, while FIG. 5B depicts mounting blocks 31 and 32 in a more forward or anterior position.

Mounting blocks 31 and 32 are secured to baseplate 30 by attaching them in angular slots 34. Slots 34 are, in one embodiment, two separate arcuate or circular slots with either a constant or variable radius. Slots 34 allow mounting blocks 31 and 32 to be individually positioned anywhere along a path of the slots. In the socket assembly 100, slots 34 are a circular path with a fixed radius. Thus, in one embodiment, a position of the struts (14, 15) (and hence the ischial seat paddle 12 and the hip paddle 13) are controlled by a position of the mounting blocks 31 and 32 on baseplate 30. The mounting blocks, in one embodiment, are secured to baseplate 30 through angular slots 34 by any suitable retention element 35 (see, e.g., FIG. 3), such as screws or bolts.

A baseplate 30 is further adjustable with respect to a lower limb attachment. The lower prosthesis connector 40, such as a pyramid connector, is mounted to a lower or distal side of baseplate 30 by radial slots 36. Radial slots 36 allow for connector 40 to be moved toward or away from the center of the baseplate, and consequently the prosthetic lower limb is operable to be positioned in a more anterior or posterior location to functionally align with the residual limb. This radial adjustability is advantageous, as it allows for ease of positioning of a prosthetic limb, since incorrect placement often results in difficult or strenuous operation of the prosthesis. In some embodiments, connector 40 can be mounted just behind the back of the leg and to the outside of the centerline of the leg to improve operation and control of the leg.

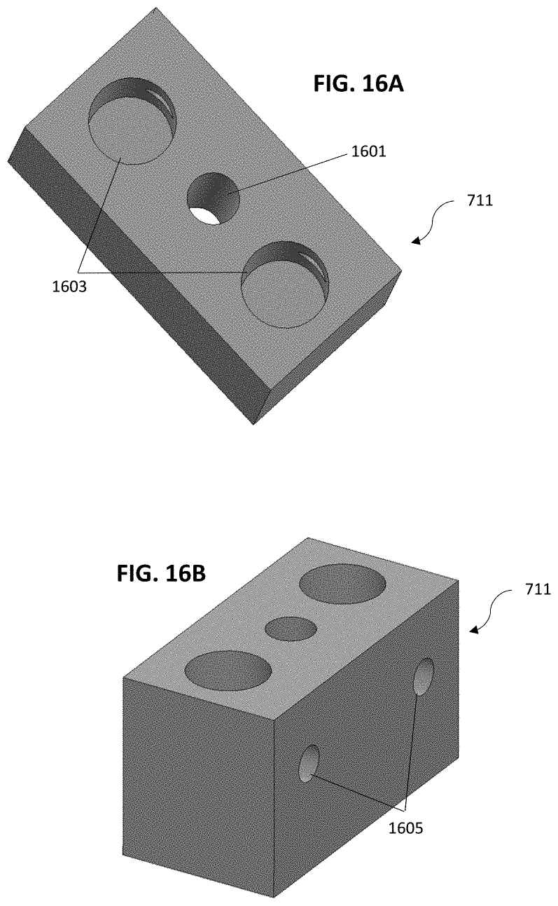

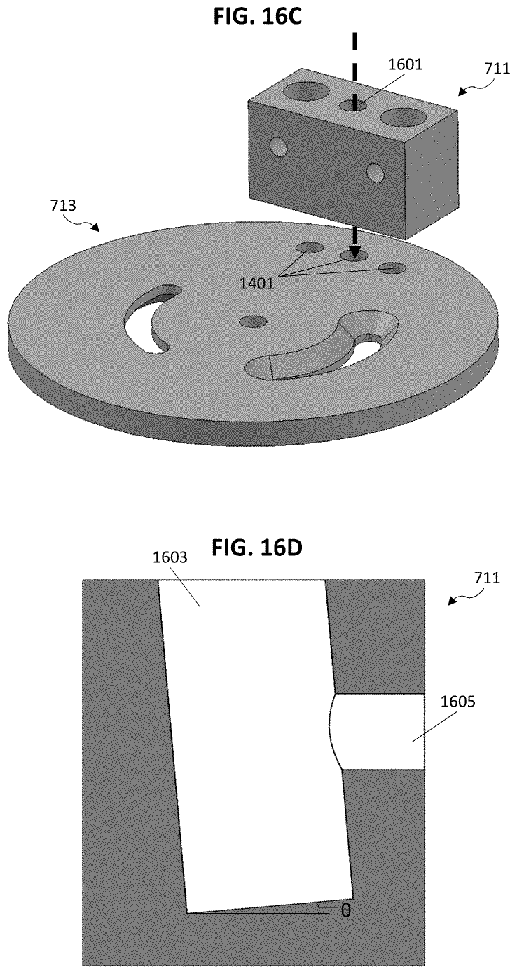

FIGS. 6A, 6B, 6C, and 6D illustrate one embodiment of a mounting block (31, 32), wherein the mounting block includes a receptacle 33. FIG. 6C illustrates a perspective view of cross section 6C-6C, and FIG. 6D illustrates a side view of cross section 6D-6D. Notably, a receptacle 33 is angled, wherein the angled slot provides the struts with an ideal angle for strength, support, and comfortability, similar to the hip rod connector illustrated in FIGS. 16A-16D.

Another advantageous feature of the socket assembly is the ability to configure the assembly for either the left or right leg. By swapping the mounting locations of the medial strut and the lateral strut, the socket assembly is operable to receive either a left or a right limb (e.g., a leg). Additionally, the socket assembly is further fully adjustable for a range of limb lengths, widths, and shapes. In some embodiments, the aforementioned features of the socket assembly are configured such that a single set of elements are operable to be adjusted to fit almost all users. Alternatively, the assembly can be provided in a variety of set element sizes (e.g., S, M, L, XL) while still adhering to the concept of a low-cost, customizable socket.

The adjustability of this device addresses, in part, issues associated with ill-fitting sockets, including but not limited to sores, bruising, discomfort and secondary muscle pain due to poor alignment.

One embodiment of this invention features a modular design with non-customized, readily available components. This is advantageous as it minimizes the amount of training needed to teach an individual to fit a user for a device, as well as the time it takes to produce the device. In part, this is due to that it avoids the time required to cast an impression and take detailed measurements, or even create a detailed three-dimensional digital profile, while still providing greater comfort to the user.

Simple fitting instructions may not require a licensed practitioner to fit the invention to a user. This is in part due to the simple adjustability of the invention. The baseplate, for example, has a wide range of adjustability and requires only commonly available tools to assemble and adjust, and does not requiring any remolding as is required for many other prostheses. Advantageously, this provides an improved fit to a user in less time with fewer resources.

Further, the socket of the present invention is operable to be used with or without a liner on the residual limb. This is advantageous, as it leads to a reduction in the amount of sweat produced, increases breathability, and simplifies the donning and doffing procedures. Furthermore, this invention does not require a pin-lock or vacuum seal to be used along with a liner, but rather it tightens around the limb in specific locations. This is advantageous because it avoids the need to put downwards pressure on the leg and socket when donning, which enables donning while sitting down, reducing the risk of fall and injury.

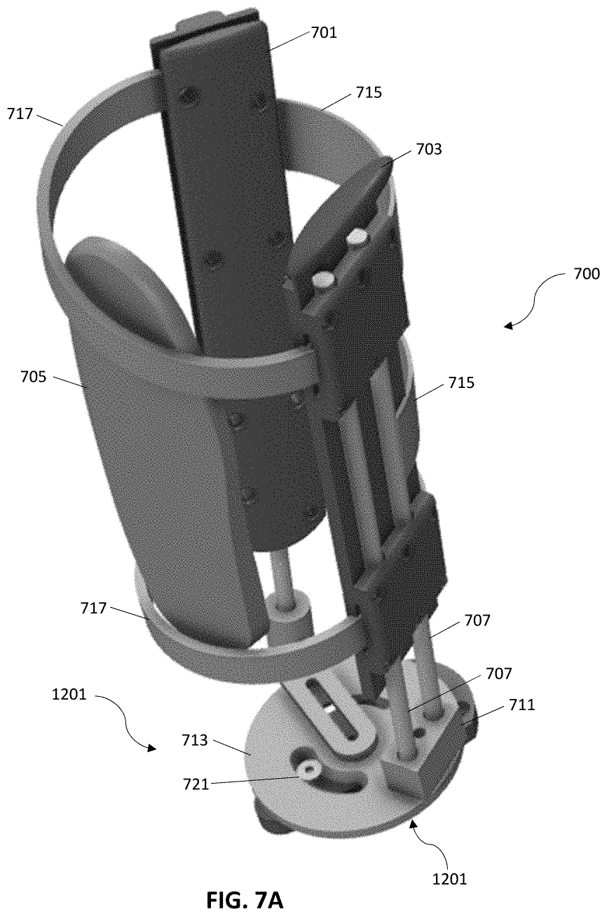

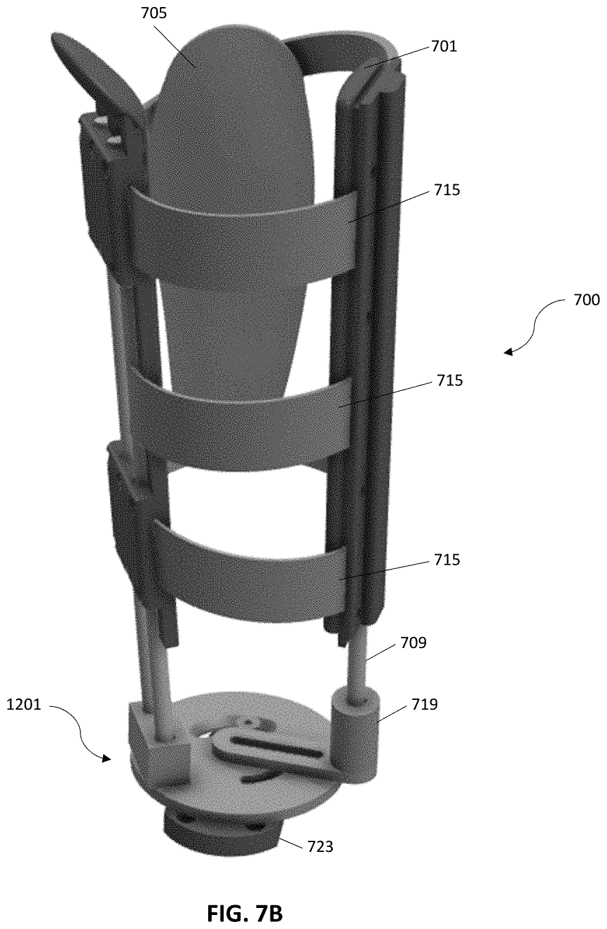

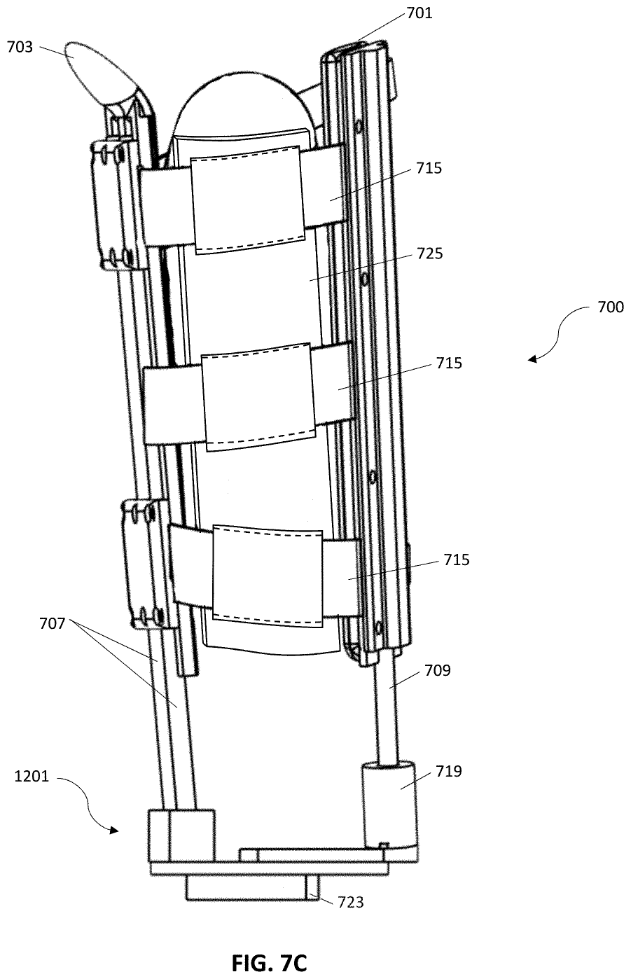

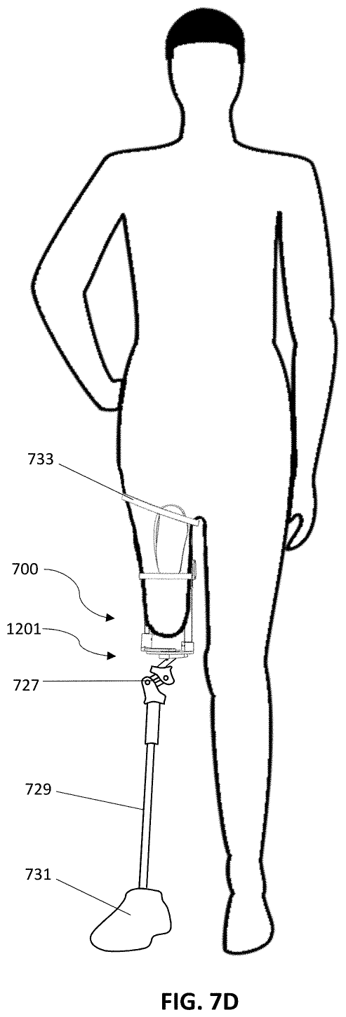

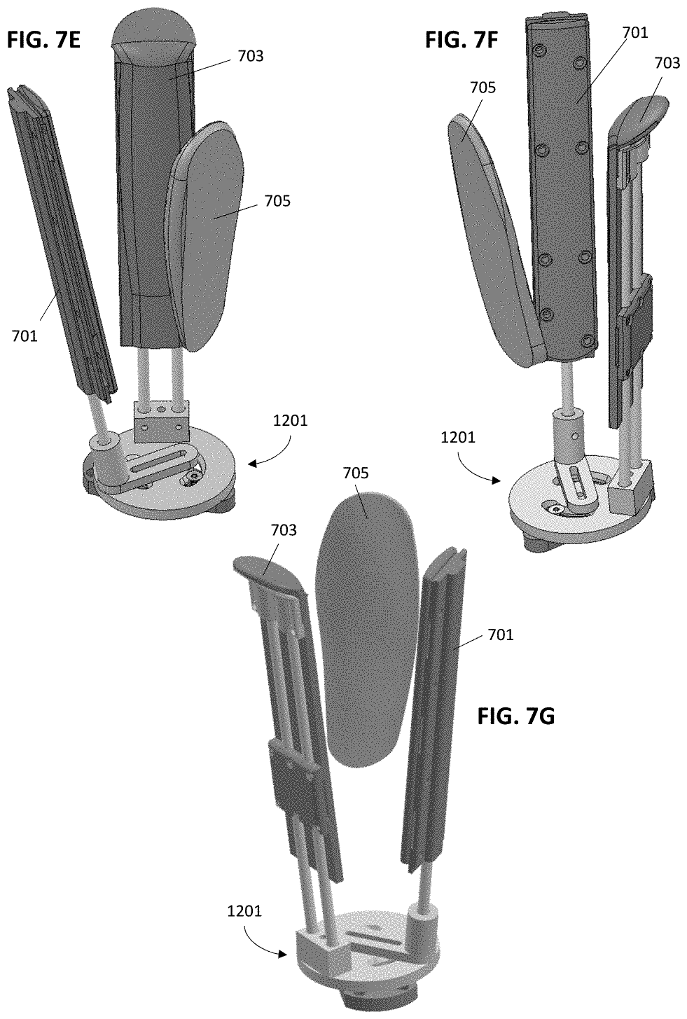

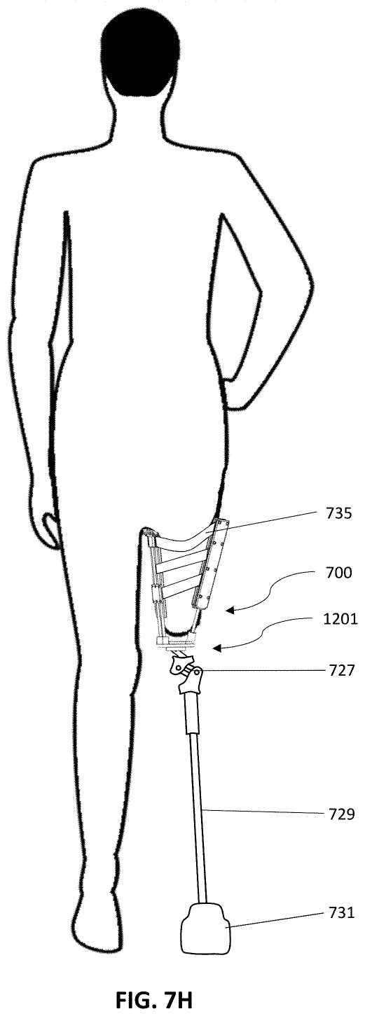

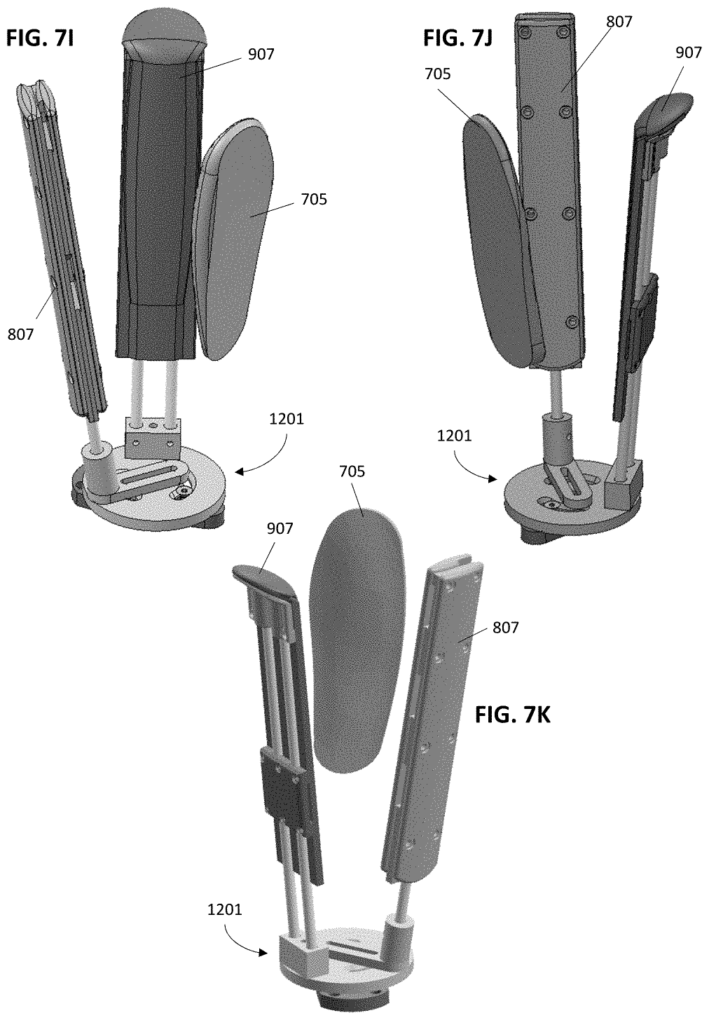

FIGS. 7A, 7B, 7C, and 7D illustrate an alternative embodiment of the socket. In this embodiment, the socket assembly is constructed to receive and secure a limb by way of three paddles: a hip paddle 701, an ischial seat paddle 703, and a femoral paddle 705. Each paddle is designed with a general contour related to pressure locations 1A, 1B, or 1C (FIG. 1), respectively. Sets of vertical support mechanisms, including ischial seat rods 707 and lateral hip rod 709, connect a proximal body attachment portion 700 to a distal baseplate assembly 1201, which is located in a distal region 702. The baseplate assembly 1201 includes a top surface and a bottom surface. The ischial seat rods 707 and lateral hip rod 709 are connected to the distal baseplate assembly 1201 by way of the ischial seat connector 711 and hip rod connector 719, respectively.

Paddles 701 and 703 are operable to translate vertically, which allows height adjustment of the assembly within an allowed range of the paddle and rod construction. In one embodiment, the ischial seat connector 711 and hip rod connector 719 are attached to the top surface of the baseplate assembly 1201. Alternatively, the paddles are set at a fixed height relative to the baseplate 713.

Rear straps 715 are attached to the hip paddle 701 and ischial seat paddle 703. In the illustrated embodiment of the socket assembly, rear straps 715 are fixedly attached to hip paddle 701, ischial seat paddle 703, and rear pad 725 in such a way that hip paddle 701, ischial seat paddle 703, and rear pad 725 are adjustable without interference. Rear straps 715 alternatively have slidable mounting points (not shown) on hip paddle 701 and ischial seat paddle 703, or, in another embodiment, are wrapped around a ischial seat rod 707 and a lateral hip rod 709 on either end of each individual strap. The paddles are additionally configured to fit between the straps and a received limb, which helps protect underlying tissue. Rear straps 715 are disposed either horizontally or at an angle in order to prevent the socket assembly from slipping. The angle is, in one embodiment, between 0 degrees and 20 degrees. In another embodiment, the angle is between 3 degrees and 7 degrees. In a preferred embodiment, the angle approximately 5 degrees. This angle is, in one embodiment, further based on a measurement from either an ischial seat rod 707 or hip rod 709 and/or is a measurement from either above or below the horizontal axis (i.e., from a top surface of the baseplate). These straps are operable to be tightened or loosened by a variety of mechanical mechanisms in order to maintain a secure fit and patient comfort, including a ratcheting mechanism, a buckle, a snap, or slider. In one embodiment, at least two straps are present. In another embodiment, the straps are set at a fixed length.

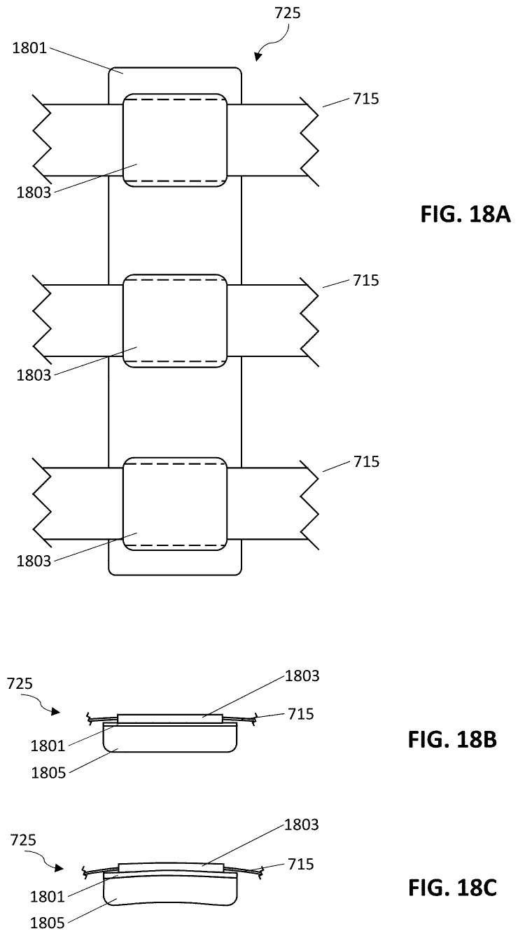

FIGS. 7A, 7B, 7C, and 7D further illustrate front straps 717 attached to hip paddle 701, ischial seat paddle 703, and femoral paddle 705. In the illustrated embodiment, the socket assembly includes front straps 717 fixedly attached to hip paddle 701, ischial seat paddle 703, and femoral paddle 705 in such a way that hip paddle 701, ischial seat paddle 703, and femoral paddle 705 are operable to be moved without interference. In an alternative embodiment, front straps 717 have slidable mounting points (not shown) on hip paddle 701, ischial seat paddle 703, and femoral paddle 705. In another embodiment, front straps 717 are wrapped around a ischial seat rod 707 and a lateral hip rod 709 on either end of each individual strap. The paddles are additionally operable to be configured to fit between the straps and the received limb, which helps protect the underlying tissue. Front straps 717 are disposed either horizontally or at an angle in order to be located above quadriceps muscles of a user and prevent the socket assembly from slipping. The angle, measured from either the ischial seat rod 707 or hip rod 709, is measured either above or below the horizontal axis. In one embodiment, the angle of a strap is between 0 and 90 degrees. In another embodiment, the angle is between 30 and 60 degrees. In a preferred embodiment, the angle is approximately 45 degrees. When more than one front strap 717 is present, the straps, in one embodiment, are non-parallel. For example, in one embodiment, the straps differ in angle between 0 and 25 degrees. In another embodiment, the straps differ in angle by approximately 15 degrees. These front straps 717 provide mechanisms for easy donning and doffing, as well as adjustment of pressure, to the user throughout the day. In one embodiment, a ratcheting mechanism includes straps composed of two shorter straps, wherein each of the two shorter straps are fixed at their distal ends to either a ischial seat rod 707 or lateral hip rod 709 and are joined at their proximal ends. Preferably, at least two straps are used in the ratcheting mechanism. In another embodiment, the straps 715 and 717 are circumferential straps. FIG. 7C further illustrates a rear pad 725, wherein the rear pad provides support and comfort to a residual limb in the socket as well as support and direction to the rear straps 715. The rear pad is illustrated in detail in FIGS. 18A-18C. In some embodiments, at least one front pad is provided, wherein the at least one front pad is attached to front straps 717, in a manner similar to how rear pad 725 is attached to rear straps 715, and wherein the at least one front pad provides support and comfort to a residual limb in the socket as well as support and direction to front straps 717.

The straps include front straps and rear straps, wherein the front and rear straps operate independently. This is advantageous, as it allows a user to tighten the prosthesis and to maintain the position of the ischial seat. In one embodiment, a cinch style securement mechanism or a commonly found strap adjuster is used in the rear because it is very secure, and the rear straps are likely to be adjusted much less often. The ratcheting straps are used in the front of the prosthesis because they are quick to use, which is advantageous as they are what will be used for adjustment of the prosthesis throughout the day.

A ratchet system allows for micro adjustment during the day, but is also more secure and durable than a material such as hook-and-loop straps. Advantageously, a user does not need to take off their prosthesis in order to change their comfort level, and adjustment can even be accomplished under clothing.

The utilization of a variety of adjustable straps in multiple locations, as demonstrated in this embodiment, is advantageous, as it provides multiple degrees of adjustability for socket fit. Varying tension adjustment is important as limb volume changes through the course of the day, and not at the same rate in each area of the limb. In one embodiment, the adjustable straps are constructed from a flat, nylon straps. In another embodiment, the straps are constructed from any rugged and/or durable material operable to secure and/or tighten elements of the socket. For example, in one embodiment, the straps are constructed out of at least one woven or non-woven materials, including nylon, cotton, wool, silk, polyester, polyethylene, or polypropylene.

Preferably, the straps, buckles, backplates, and other securing elements of the socket are constructed with durable and/or rugged materials that eliminate issues caused by systems with many, smaller parts and complex mechanics that are prone to breaking during prosthetic usage.

Preferably, at least one strap attached to the hip paddle and the ischial seat paddle is positioned to be worn high on a leg of the user, wherein a length and angle of the front strap is constructed to be secured around an upper side of the femoral triangle of a body and near to the inguinal ligament such that a front strap nearly follows a groin line of the user above the quadriceps, and wherein a rear strap is positioned to support the ischial seat and gluteal fold. This positioning of at least one front strap and rear strap pair allows for an advantageous distribution of weight, wherein a strap positioned closer to a groin of the user allows for a comfortable dispersion of pressure to inguinal ligaments in the front, and wherein a corresponding rear strap support the gluteal fold in the rear. In one embodiment, the straps are secured to a top of the hip paddle and a top of the ischial seat paddle. In another embodiment, the hip paddle and/or the ischial seat paddle extends past attachments points for the straps. Straps are, in one embodiment, attached directly to the hip rod or the ischial seat rods through any mechanical, physical, or chemical means (e.g., by wrapping around a rod, by adhesive, by ratchets, by snaps, by latches, or by screws). In another embodiment, the straps are attached to front and/or rear plates of the hip panel and/or the ischial seat panel through any mechanical, physical, or chemical means. The straps are attached to an interior and/or an exterior of the hip or ischial seat paddles. In one embodiment, the rods, the paddles, and/or the backplates include reversible attachment points for the straps for both left-leg and right-leg embodiments. For example, in one embodiment, the rods and/or paddles include two ratcheting mechanisms, wherein a front ratcheting mechanism is positioned with a first angle corresponding to an inguinal ligament, and wherein a rear ratcheting mechanism is positioned with a second angle corresponding to a gluteal fold. The ratcheting mechanism is adjustable as a whole to a left-leg position or a right-leg position, wherein the ratcheting mechanism is translatable and/or rotatable, and wherein the ratcheting mechanism further includes detents that secure the ratchets in position for left-leg use or right-leg use. In another embodiment, the ratcheting mechanism rotates freely when not secured by a pin, screw, or other means, and wherein an angle of the straps is continuously adjustable.

FIG. 7D illustrates an attachment of the socket to an individual's residual limb, according to one embodiment of the present invention. The proximal body attachment portion 700 is secured around a residual limb and is further secured to the distal baseplate assembly 1201. The distal baseplate assembly 1201 is further secured to a prosthetic knee 727, lower leg 729, and foot 731 which all work to provide support to a user. The front strap 733 is preferably attached to a top of the hip paddle and the ischial seat paddle to enable the strap to be adjusted secured to a user's body just below the inguinal ligament.

FIGS. 7E, 7F, and 7G illustrate three different rotated views of a socket assembly without straps according to another embodiment of the present invention. In the illustrated embodiment, the socket includes a hip paddle 907, an ischial seat paddle 907, and a baseplate assembly 1201.

FIG. 7H illustrates an attachment of the socket to an individual's residual limb, according to one embodiment of the present invention. The proximal body attachment portion 700 is secured around a residual limb and is further secured to the distal baseplate assembly 1201. The distal baseplate assembly 1201 is further secured to a prosthetic knee 727, lower leg 729, and foot 731, which all work to provide support to a user. The rear strap 735 is preferably attached to a top of the hip paddle and top of the ischial seat paddle to enable the strap to be adjusted secured to a user's body just below the gluteal fold.

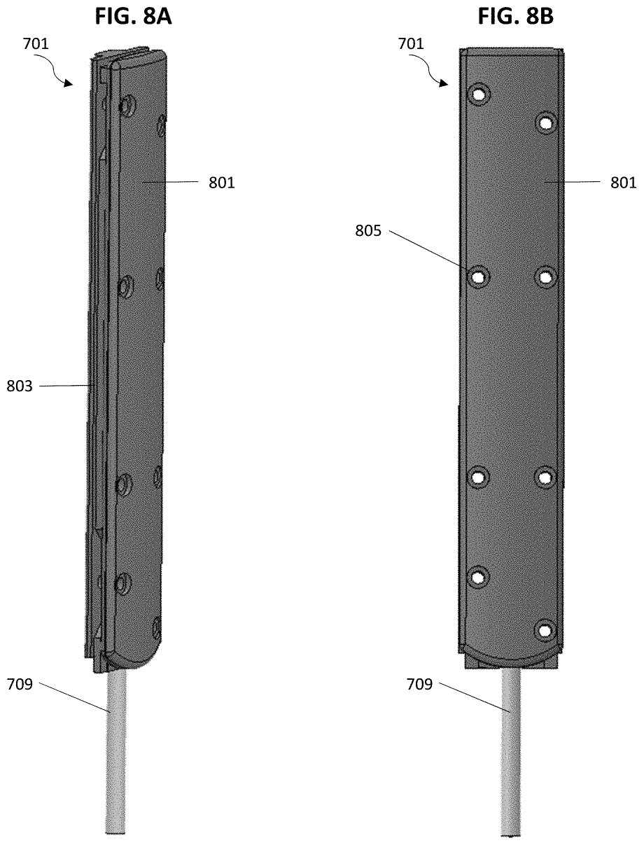

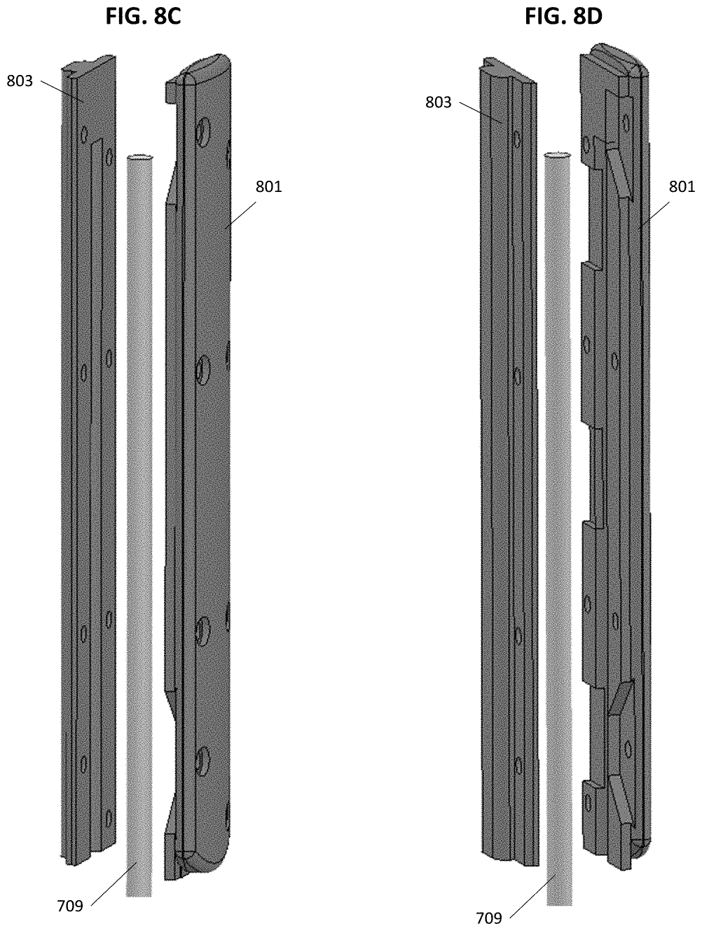

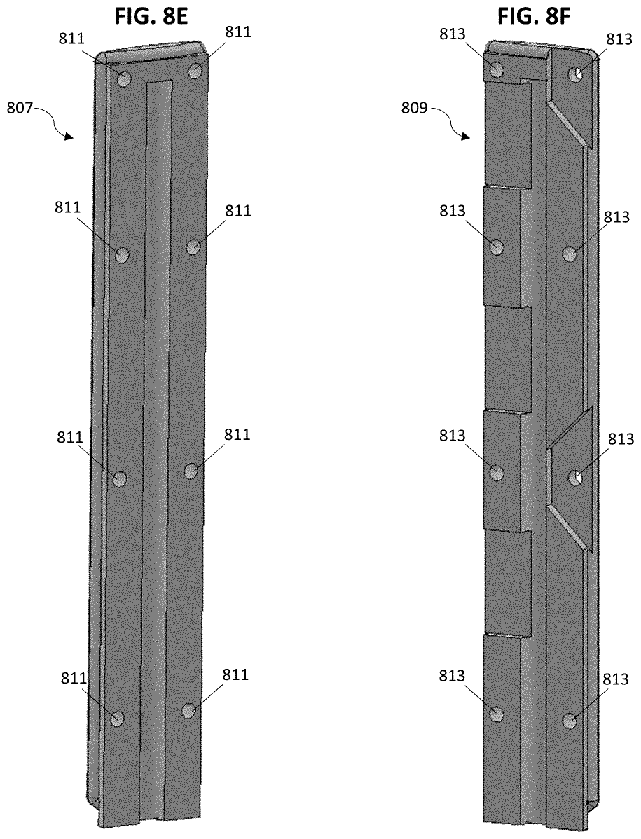

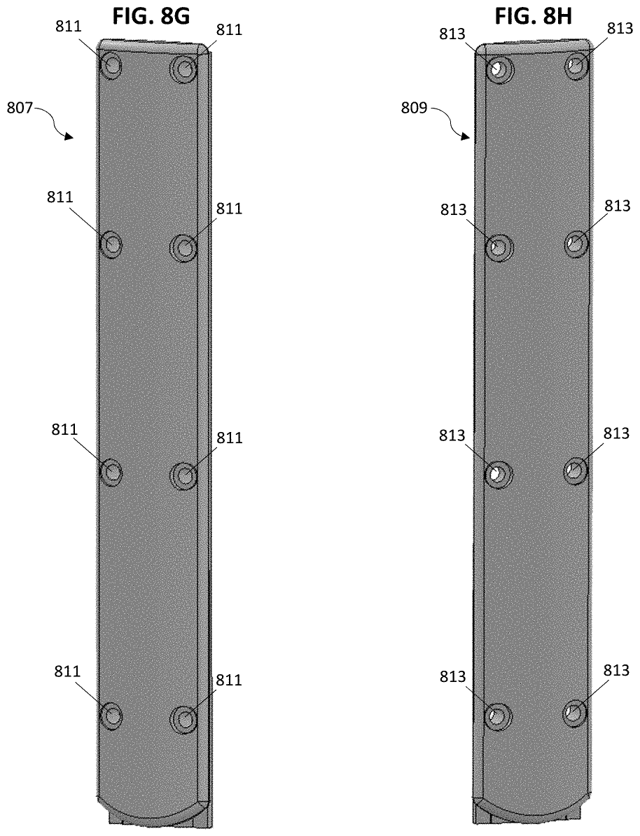

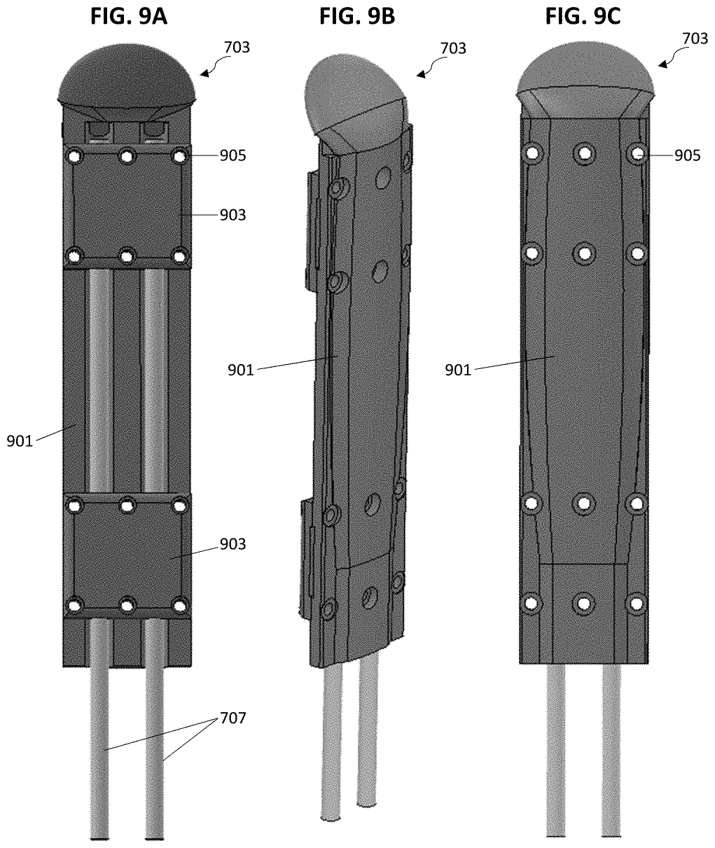

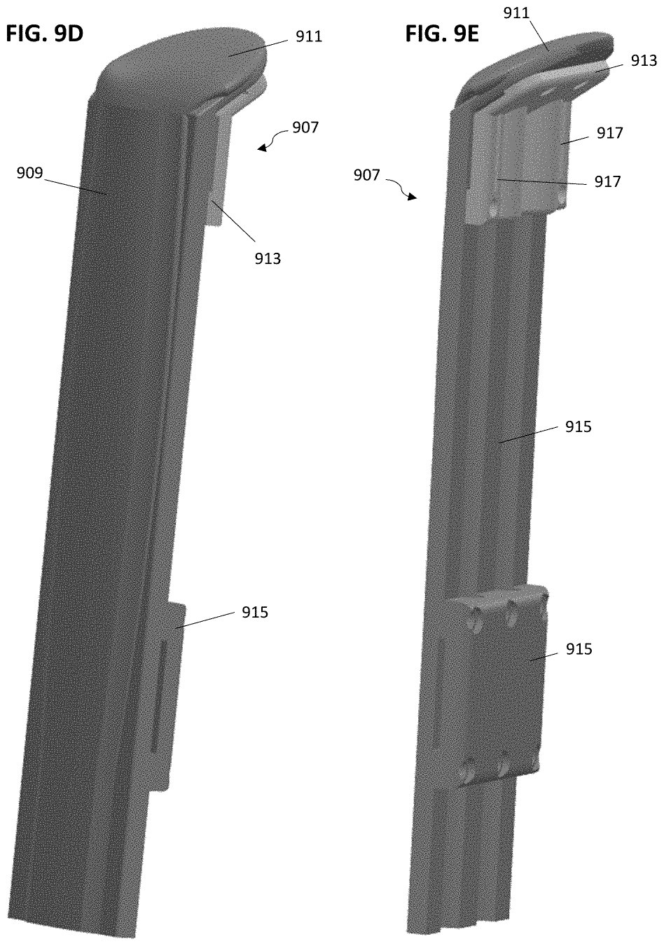

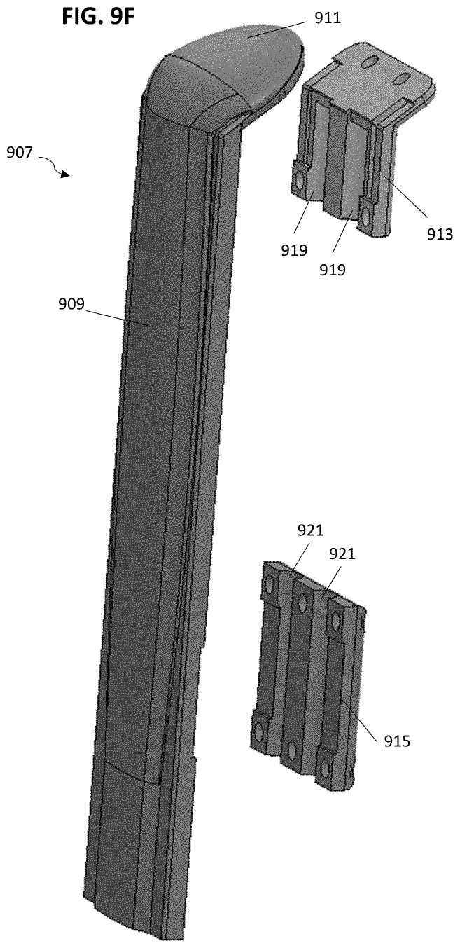

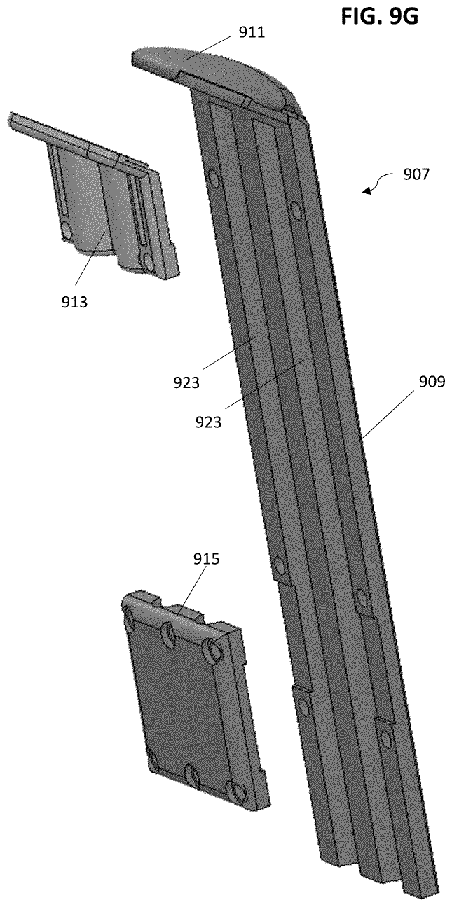

FIGS. 7I, 7J, and 7K illustrate three different rotated views of a reversible socket assembly without straps according to another embodiment of the present invention. In the illustrated embodiment, the socket includes a reversible hip paddle 907, reversible ischial seat paddle 907, and a reversible baseplate assembly 1201, each of which are illustrated and described in detail with respect to FIGS. 8E-8H and 9D-9G

FIGS. 8A and 8B illustrate perspective and front views, respectively, of a hip paddle 701. The hip paddle 701 is constructed from at least hip plate 801 and one or more backplates 803. Hip plate 801 and backplates 803 function to compress and secure the hip rod 709 and straps (715,717) within the hip paddle 701 and provide support during prosthetic usage. The hip paddle backplates 803, in one embodiment, are operable to be disconnected and placed at different distances along the hip rod 709 relative to the distal baseplate assembly 1201 in order to provide customizability in accommodating variations in residual limb sizes and to extend a length of the socket. In one embodiment, the hip plate 801 and backplates 803 are connected via screws, bolts, or staples, which pass through holes 805. Alternatively, the hip plate 801 and the backplate 803 are joined via snap fit, latch, or any other attachment mechanism known in the art of mechanical design.

FIGS. 8C and 8D illustrate exploded views of the hip paddle, including a backplate 803 and a hip plate 801. These figures illustrate the backplate 803 and hip plate 801, wherein the backplate 803 and the hip plate 801 secure opposite sides of the hip rod 709 and extend past an end of the hip rod 709.

In one embodiment, the hip plate 801 and the backplate 803 are mirrored embodiments of each other such that the hip paddle is reversible and/or is operable to be used in both left-leg and right-leg embodiments. For example, in one embodiment, the hip plate 801 and the backplate 803 both have a smooth surface for contact with a body of a user.

FIGS. 8E and 8F illustrate rear views of another embodiment of a backplate 807 and a hip plate 809, wherein the backplate 807 and the hip plate 809 include holes (811, 813) that are positioned along a top of the backplate 807 and hip plate 809 and are positioned symmetrically along each component. The backplate holes 811 and the hip plate holes 813 are correspondingly constructed such that retaining elements (e.g., bolts) are operable to extend through the holes (811, 813) and secure the components together. Preferably, the positioning of the holes (811, 813) in the illustrated embodiment allows for positioning of the straps along the inguinal ligament and the gluteal fold for increased comfort and support. Additionally, the symmetric positioning allows for reversibility, wherein the two panels are operable to be rotated for use on a left leg or a right leg. FIGS. 8G and 8H illustrate front views of the backplate 807 and the hip plate 809 embodiment illustrated in FIGS. 8E and 8F. Notably, a surface of each of the paddles is smooth to allow reversibility, wherein a backplate is attached with an outer surface facing an inside of the socket (for contact with a user's body), and a hip plate is attached with an outer surface facing an outside of the socket, and wherein comfort is maintained through smooth surfaces that contact a user's limb. This construction allows for strap angles and positions of both front and rear straps to be correctly positioned between right-leg and left-leg embodiments.

FIGS. 9A, 9B, and 9C illustrate rear, perspective, and front views, respectively, of the ischial seat paddle 703, which, in one embodiment, is intended to be the primary load-bearing mechanism of the socket. Ischial seat paddle 703 is constructed from at least an ischial plate 901 and multiple backplates 903. The backplates 903 and ischial plate 901 function to compress the two or more ischial seat rods 707 to prevent vertical motion between the seat and supporting bars during use. The backplates 903 and ischial plate 901 also function to secure the straps (715, 717). Alternatively, the backplates 903 are disconnected, wherein each of the backplates 903 are operable to be placed at different distances along the seat rods 707 relative to the distal baseplate assembly 1201 to provide customizability in accommodating variations in residual limb sizes. In one embodiment, the ischial plate 901 and backplates 903 are connected via screws, bolts, or staples, which pass through holes 905. Alternatively, the ischial plate 901 and the backplates 903 are joined via snap fit, latch, or any other attachment mechanism known in the art of mechanical design. Preferably, the backplates 903 of the ischial seat paddle 703 and screw holes of the hip paddle 701 are positioned to eliminate stress and strain on the assemblies during use.

FIGS. 9D and 9E illustrate another embodiment of an ischial seat paddle 907, wherein the ischial seat paddle 907 includes backplates (913, 915) that are positioned higher than the previous embodiment. Preferably, a first backplate 913 is positioned underneath a seat 911 of the ischial seat paddle 907, wherein the seat 911 provides vertical support to a user, and wherein the first backplate extends along a vertical portion 909 of the ischial seat paddle 907 and below the seat 911. The first backplate 913 includes strap channels 917, wherein the strap channels 917 extend along both the vertical and non-vertical sections of the first backplate 913. This strap position, compared to the previously illustrated embodiments, increases support and stability for the user by allowing a strap position that both supports the gluteal fold as well as follows a portion of a user's body that is parallel to the inguinal ligament, providing more control and security. In the illustrated embodiment, a second backplate 915 is additionally attached along a lower half of the paddle and provides further security to the ischial seat rods. In another embodiment, the second backplate 915 is positioned higher or lower along a vertical portion 909 of the ischial seat paddle 907. In one embodiment, the ischial seat paddle 907 includes threaded holes or other means of attachment for at least one backplate (e.g., 913, 915). However, in another embodiment, the ischial seat paddle 907 includes sliders, rails, slots, chains, or other similar methods of allowing the at least one backplate to be adjustable vertically, horizontally, or away from the ischial seat paddle. In one embodiment, the backplates are removable or adjustable to allow for straps to be easily inserted and removed from the prosthetic socket.

FIG. 9F illustrates a front exploded view of the ischial seat paddle 907 with higher strap positioning according to one embodiment of the present invention. In the illustrated embodiment, the first backplate 913 includes end slots 919 and the second backplate 915 includes channels 921 for receiving and securing at least one ischial seat rod. The end slots 919 secure ischial seat rods in place between the first backplate 913 and the vertical portion 909 and/or seat 911 of the ischial seat paddle 907. The end slots 919 further convey vertical forces to the seat 911 of the ischial seat paddle 907. FIG. 9G illustrates a rear exploded view of the ischial seat paddle 907, including vertical channels 923 integrated within a vertical portion 909 of the ischial seat paddle 907, wherein the vertical channels 923 are a second corresponding half to the end slots 919 and the channels 921 of the first backplate 913 and the second backplate 915, respectively. The vertical channels 923 secure at least one ischial seat rod in place to prevent horizontal and vertical movement.

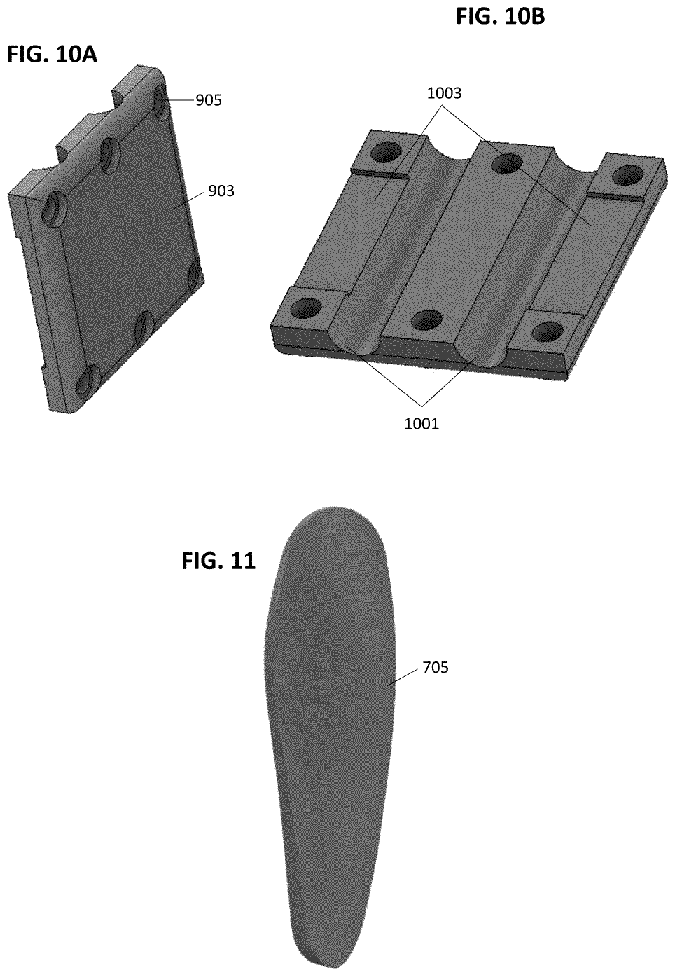

FIGS. 10A and 10B illustrate additional embodiments of a backplate 903. On one face, holes 905 are countersunk to allow the connecting component (e.g. a bolt) to sit flush along the surface. On the reverse side, channels 1001 allow the backplates 903 to have more contact area with ischial seat rods 707 when attached to ischial plate 901. Strap channels 1003 provide room for straps to be secured between the ischial plate 901 and backplates 903.

In one embodiment, the straps are wrapped around the support rods and are further secured between the main plate and backplates. Compared to other devices, which often secure the straps with stitching or by folding them between layers of fabric each time the socket is to be donned, this method is advantageous because it is both more secure and provides more support, but the socket still provides a simple solution for replacing components if the need arises. In the case of ratchet straps, in one embodiment, a ribbed side of the strap (e.g. the female end) is secured directly between the two sides of a paddle. This is advantageous in avoiding gaps in securement as well as eliminating any potential interference with soft tissue.

FIG. 11 illustrates the femoral paddle 705 without any mounting hardware. In a preferred embodiment, the femoral paddle 705 is contoured similar to the quadriceps muscle to maximize patient comfort.

The use of two fixed paddles (the ischial seat paddle 703 and hip paddle 701) and one or more floating paddles, such as the femoral paddle, is advantageous to methods using three or more separate groups of fixed struts, as it provides increased adjustability, fewer opportunities to pinch the skin of the user, and pressure can more easily be tailored to the user. Further, in part due to the less rigid shape, there are less components to physically adjust for the user. In one embodiment of the present invention, there is no rigid item connecting any of the rods or struts, which allows for further adjustability in accommodating a variety of residual limb sizes.

This nonrestrictive shape is also advantageous because it provides relief and airflow compared to a traditional socket. Further, support components allow for more force to be applied closer to a bone of the residual limb preventing the bone from shift in the socket during use.

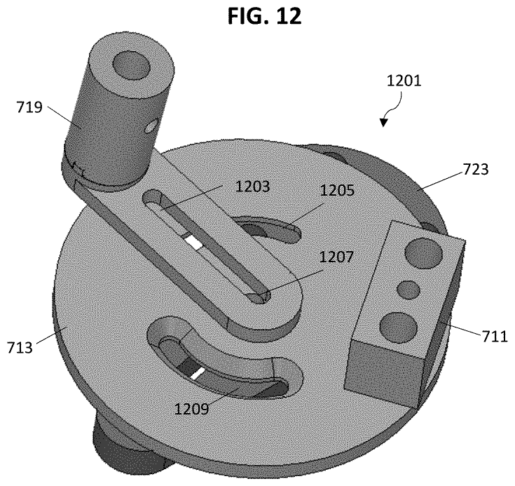

FIG. 12 illustrates one embodiment of a baseplate assembly 1201 of the prosthetic which includes at least four distinct parts, including a knee connector 723, a hip rod connector 719, an ischial seat connector 711, and a baseplate 713. The hip rod connector 719 is constructed to secure the hip rod, the ischial seat connector 711 is constructed to hold the ischial seat rods, and the knee connector 723 is constructed to attach to an external support prosthetic, such as a prosthetic knee with a prosthetic leg and foot (see FIG. 7D). The support prosthetic is preferably attached to the knee connector 723 via a pyramid connector; however, the support prosthetic is alternatively attached via any other joint or connector known in the art of mechanical design. In one embodiment, the hip rod connector 719 is attached to the baseplate 713 via a hip rod connector slider 1203 and a center pin. The center pin extends through the hip rod connector slider 1203, through a center bore 1207, and through a slider of the knee connector 723. The center pin serves to secure the hip rod connector in place when tightened as well as provide circumferential motion around the baseplate 713 within the hip rod connector slider 1203 when loose. A rotational pin serves to control the circumferential range of motion of the hip rod connector, which provides further customizability for easy adjustment and fit for a variety of end users by way of continuous, rather than discrete, adjustability, wherein the rotational pin provides motion within a rotational slider slot 1205. Thus, a hip rod connector 719 that is loosely attached to the baseplate 713 is operable to extend outwards and inwards with radial movement governed by the center pin as well as move angularly around the baseplate 713 governed by the rotational pin and the rotational slider slot 1205, which provides further customizability for easy adjustment and fit for a variety of end users by way of continuous, rather than discrete, adjustability. When tightened via a bolt, threaded bore, or other attachment mechanism, the parts do not freely move. Notably, a pin is any pin, screw, bolt, or other attachment means that is operable to both allow movement within a slider as well as secure the parts together. Other washers, spacers, or additional mechanical means of attaching, securing, and allowing movement are alternatively used with the prosthetic. Preferably, the baseplate 713 is constructed with a knee-baseplate slider slot 1209, which advantageously renders the baseplate 713 and baseplate assembly 1201 reversible. For example, the embodiment illustrated in FIG. 12 is assembled for a right leg assembly. In one embodiment, the baseplate 713 is symmetric for use in left or right leg embodiment, wherein a 180 degree rotation or a mirror of the orientation allows the ischial seat connector 711, the hip rod connector 719, and the knee connector 723 to be secured and prepare the baseplate assembly 1201 and the proximal body attachment portion 700 for use on the left leg instead of the right.

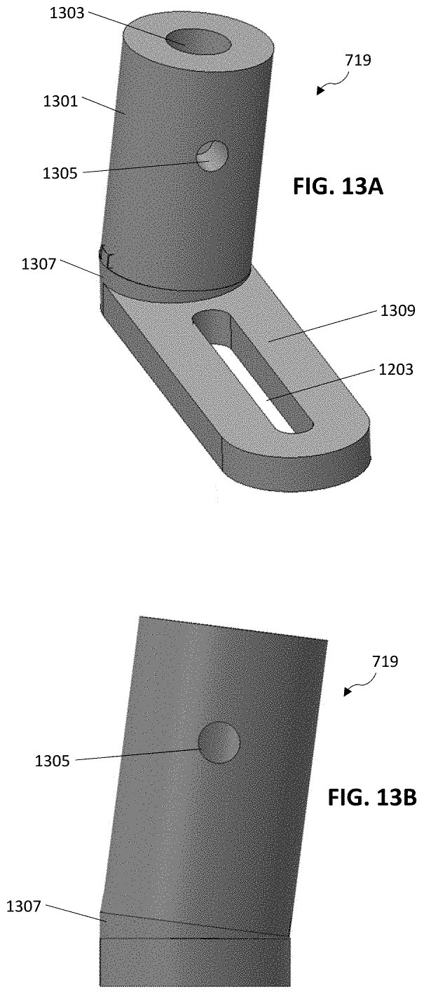

FIG. 13A illustrates one embodiment of the hip rod connector 719, wherein the hip rod connector includes an extruded foot 1309 with a hip rod connector slider 1203, a cylinder 1301, a hip rod bore 1303 for insertion of a hip rod, a securing bore 1305 for insertion of a securing pin, and at least one angled shim 1307. Preferably, a hip rod is inserted via the hip rod bore 1303 and is secured in the cylinder 1301 via a pin. In one embodiment, the cylinder 1301 is fully hollow, wherein the hip rod bore 1303 extends through the cylinder 1301 to the shim 1307. In another embodiment, the hip rod bore 1303 extends through approximately 80% and 100% of the cylinder 1301. In a further embodiment, the hip rod bore 1303 extends through approximately 50% to 100% of the cylinder 1301.

Advantageously, the hip rod connector 719 includes at least one angled shim 1307, wherein the angled shim 1307 provides more comfortable prosthetic usage by tilting the cylinder 1301 towards a rear of the received limb. In one embodiment, the angle of displacement is between 2.5 degrees and 10 degrees. In another embodiment, the angle is between 4 degrees and 8 degrees. In yet another embodiment, the angle of displacement is between 5 degrees and 8 degrees. In a preferred embodiment, the angle is approximately 5 degrees. The shim is either constructed as a single part with an angled surface or includes multiple angled spacers or angled shims stacked to provide a preferred angle of displacement. FIG. 13B illustrates a front view of the hip rod connector 719, including the securing bore 1305 and the at least one shim 1307. In one embodiment, the securing bore 1305 is positioned on an inside of the hip rod connector 719. In an alternative embodiment the securing bore 1305 is positioned at any position around the cylinder 1301. In a further embodiment, multiple securing bores are positioned around the cylinder 1301 and provide additional security for an inserted hip rod. In another embodiment, the shim 1307 is integral with the hip rod connector, wherein the shim 1307 is constructed integrally with cylinder 1301 and/or the extruded foot 1309.

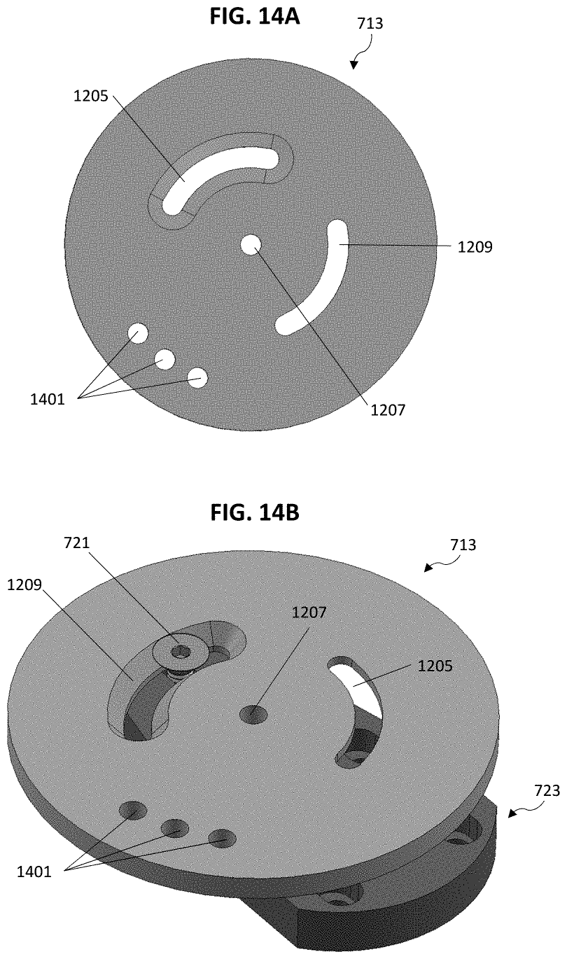

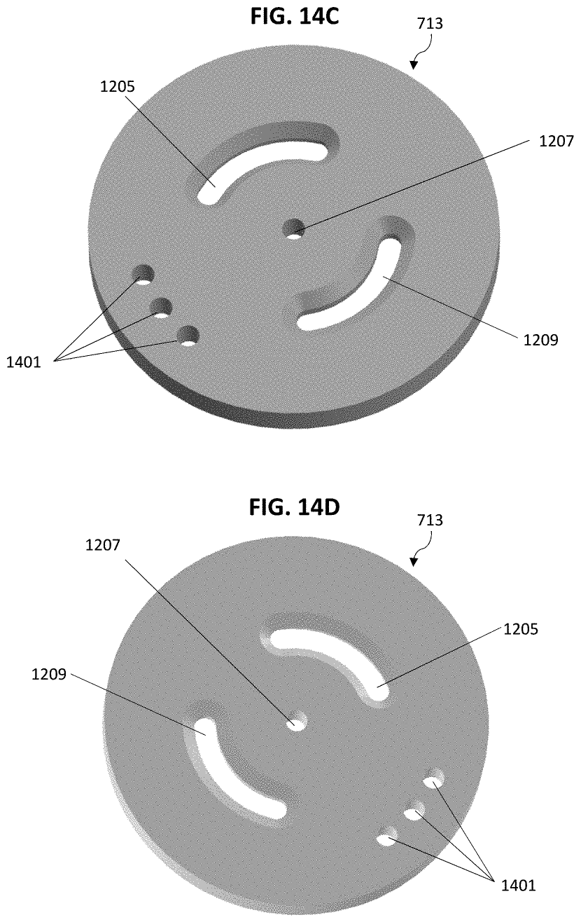

FIG. 14A illustrates a bottom view of the baseplate 713, wherein the baseplate 713 provides attachment points and alignment points for each of the elements of the baseplate assembly, including slider slots (1205, 1209), ischial seat connector bores 1401, and a center bore 1207. The slider slots (1205, 1209) are constructed with countersinks to allow for ease of movement and a flat profile with any attached pins. In one embodiment, the countersinks are constructed on opposite sides, wherein a bottom element (e.g., the knee connector) is connected to a slider with a countersink on a top side of the baseplate, and wherein a top element (e.g., the hip rod connector) is connected to a second slider with a countersink on a bottom of the baseplate. FIG. 14B illustrates a perspective view of the baseplate 713, wherein a knee connector pin 721 is secured within the knee-baseplate slider slot 1209. The head of the knee connector pin 721 is substantially parallel to the top surface of the baseplate 713. In one embodiment, the baseplate is reversible, wherein the prosthetic is operable to be reconfigured from right leg embodiment to a left leg embodiment using the same parts. For example, the baseplate 713 is constructed with slider slots that are arranged in symmetric or mirrored positions, wherein the baseplate is operable to be used in both a left leg and a right leg embodiment, and wherein the rods are operable to attach to the plates in similar, mirrored, or rotated embodiments and/or wherein the rods are operable to attach to a baseplate in a mirrored or rotated orientation.

FIG. 14C illustrates another embodiment of the baseplate 713, wherein countersinks are provided on both sides of the slider slots (1205, 1209), and wherein any mirrored or rotated orientations provide the same level of flush assembly as in a non-reversible embodiment. FIG. 14D illustrates an underside of the baseplate 713 embodiment illustrated in FIG. 14C. In a further embodiment, the baseplate includes multiple slider slots (1205, 1209) for customization, adjustability, and use in left or right leg orientations. In another embodiment, slider slots (1205, 1209) are adjustable, wherein a length, position, and/or width of the slider slots (1205, 1209) are operable to be extended, translated, and/or expanded by way of a retractable plate, internal slider or track, and/or any other mechanical means known in the art of mechanical design. In one embodiment, the baseplate 713 is between ¼ and ¾ inches (6.35 and 19.05 millimeters) thick. Preferably, the baseplate 713 is ½ inches (12.7 millimeters) thick and is constructed from aluminum. Notably, the thickness and material of the baseplate has a significant effect on the weight of the socket as well as the ability for the baseplate to be reversible. Though experimentation, it has been determined that a baseplate that is ½ inches (12.7 millimeters) thick and constructed from aluminum is one embodiment that balances both weight and the thickness required to provide chamfers and/or counter-sinks on a top and bottom of the slider slots. In another embodiment, a first baseplate is operable to be secured on top of a second baseplate, wherein the first baseplate includes a slider slot and attachment points for a right leg embodiment, wherein the second baseplate includes a slider slot and attachment points for a left leg embodiment, and wherein the combined baseplate is reversible and is operable to function for both right-leg and left-leg prosthetics. Securing for the baseplates occurs via any physical, mechanical, or chemical means known in the art, including by latches, snaps, threaded means, screws, bolts, or adhesives. The baseplate is, in one embodiment, constructed from any other material known in the art of prosthetics, such as steel, titanium, plastic, or carbon fiber.

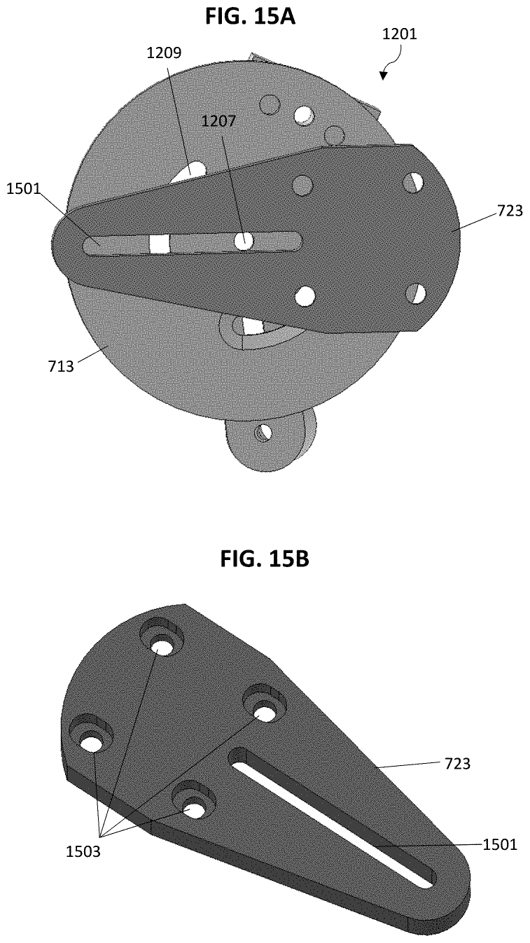

FIG. 15A illustrates a bottom view of the baseplate assembly 1201, wherein the knee connector is positioned such that an inserted knee connector pin extends from the knee-baseplate slider slot 1209 through the knee connector slider 1501. A center pin extends through the center bore 1207 through the knee connector slider 1501. Thus, the knee connector 723, when loosely attached to the baseplate 713, is operable to extend outwards and inwards with radial movement governed by the center pin as well as move angularly around the baseplate 713 governed by the knee connector pin and the knee-baseplate slider slot 1209, wherein the knee connector slider 1501 and knee-baseplate slider slot 1209 provides continuous rather than discrete adjustability in order to tailor a perfect fit for the user. The knee connector is advantageously adjustable such that a joint (e.g., a pyramid joint), an artificial knee (704), and/or a center of mass of the prosthetic as a whole is positioned directly below the ischial seat region 1B (FIG. 1A). When tightened via a bolt, threaded bore, or other attachment mechanism, the parts do not freely move. FIG. 15B illustrates a top view of the knee connector 723, wherein connection points 1503 provide for attachment to the at least one support prosthetic. In one embodiment, screws are inserted via the connection points 1503 and attach a pyramid connector for attachment to an artificial limb.

FIG. 16A illustrates a top perspective view of the ischial seat connector 711, wherein the ischial seat connector 711 includes two ischial seat bores 1603 for receiving the ischial seat rods and an attachment bore 1601 for securing the ischial seat connector 711 to the baseplate. The ischial seat paddle and the ischial seat rods are designed to support a majority of a wearer's weight during usage. Thus, in the illustrated embodiment, the prosthetic is designed with an ischial seat connector 711 that is secured in position and cannot move laterally or vertically relative to the baseplate face. The secured ischial seat connector 711 allows for ease of fitting and installation, wherein the ischial seat paddle is positioned first, and wherein other elements of the prosthetic (e.g., the hip paddle, the knee connector) are positioned relative to the ischial seat paddle. The secured ischial seat connector avoids the problem of constant resetting each of the prosthetic elements to provide a perfect fit. Instead, the current design allows for quick, easy installation without professional reconstruction of each part of the prosthetic. Preferably, the ischial seat connector 711 is rotatable around an ischial seat connector pin when loosened, wherein the ischial seat connector pin extends through the attachment bore 1601 and through one of the ischial seat connector bores of the baseplate, wherein the connector pin is operable to be secured at a continuous, custom angle, which provides continuous rather than discrete adjustability in order to tailor a more customized, comfortable, and secure fit for a user. When tightened or secured, the ischial seat connector is prevented from rotating (either via friction or any other mechanical means). FIG. 16B illustrates a side perspective view of the ischial seat connector 711, wherein securing bores 1605 are constructed to receive pins or bolts to secure the rods within the ischial seat connector 711.

FIG. 16C illustrates an exploded view of the ischial seat connector 711 and the baseplate 713, wherein the ischial seat connector 711 is positioned over the ischial seat connector bores 1401. Notably, the ischial seat connector 711 is operable to be positioned and attached via any of the ischial seat connector bores 1401, wherein a pin extends through the attachment bore 1601 through one of the ischial seat connector bores 1401. This provides highly adjustable angles and positioning for the ischial seat paddles and rods. Preferably, the ischial seat connector is operable to rotate around a loosened pin and is prevented from rotating when the pin is tightened (for example, with a nut).

FIG. 16D illustrates a section view of the ischial seat connector at a plane bisecting an ischial seat bore 1603. Preferably, the ischial seat bore 1603 is angled to provide a secure fit and improved comfort of an ischial seat paddle. The angle θ is, in one embodiment, between 2.5 degrees and 9 degrees. In another embodiment, the angle θ is between 4 degrees and 7 degrees. In a preferred embodiment, the angle θ is approximately 5 degrees. In the illustrated embodiment, the ischial seat bore 1603 extends through approximately 80% of the ischial seat connector 711. However, in another embodiment, the ischial seat bore 1603 extends completely through the ischial seat connector 711, and a shim or other spacer is inserted to provide an angular platform for an ischial seat paddle to rest on. The securing bore 1605 is illustrated as being substantially parallel to the top and bottom face of the ischial seat connector 711; however, in another embodiment the securing bore 1605 is angled. For example, in one embodiment, the ischial seat bore is angled normal to the ischial seat bore such that a pin is inserted normal to an ischial seat rod. The securing bore 1605 is further illustrated as extending to the ischial seat bore 1603. However, in an alternative embodiment, the securing bore extends past the ischial seat bore 1603 and/or extends through a whole of the ischial seat connector 711. Alternatively, the pin is operable to be secured by a cross bar, nut, adhesive, or other mechanical, physical, or chemical method of attachment known in the art of mechanical design.

In one embodiment, the ischial seat connector is the first component fitted to a user during usage. User fitting begins with the ischial seat, as it provides the most support and is also the cause of the most discomfort in traditional sockets. The ischial seat is placed at a height that is ideal for the user, and then it is fixed in place to the baseplate by the ischial seat connector. The ischial seat connector is operable to then be rotated about its axis and tightened down. All other components are then adjusted relative to the ischial seat. This is advantageous in providing a more patient specific fit without the need to have custom molded parts made. Securing the ischial seal allows for alignment of all other components at angles that match a user's body and preference, which maximizes patient comfort and mobility without the need to determine every relative location of the multiple components.

For both the hip rod connector and ischial seat connector, the components are operable to be constructed with various sizes and shapes and components (e.g., various sized shims 1307 and alternately sized parts (e.g. connectors 709 and 711)) based on a desired fit for a user. This is advantageous, as the socket is operable to be constructed from prefabricated parts, rather than customizing every part to a specific user.

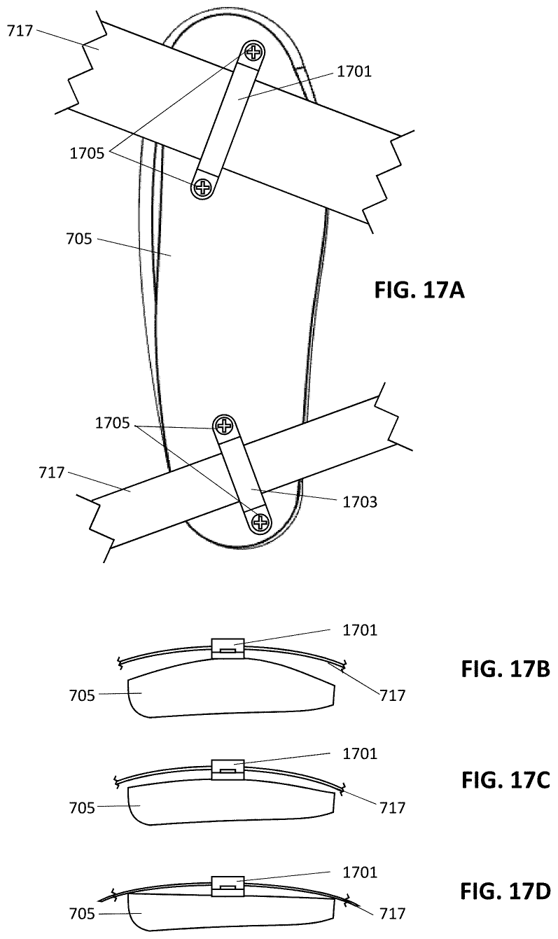

FIGS. 17A, 17B, 17C, and 17D illustrate the femoral paddle 705, which, in one embodiment, is attached to the proximal body attachment portion 700 in a different manner than an ischial seat paddle 703 and a hip paddle 701. In the illustrated embodiment, femoral paddle 705 is slidably attached to front straps 717. This construction provides femoral paddle 705 freedom of movement for placement over the femoral region 1A (FIG. 1). Femoral paddle 705 is operable to translate vertically, slide circumferentially (i.e., around perimeter of the leg), or rotate in a vertical plane to provide continuous rather than discrete adjustability in order to tailor a more customized, comfortable, and secure fit for a user. The femoral paddle 705 further includes a curvature that is convex with respect to a contacted region of a received limb. The convex shape aids in correct ergonomic placement of the paddle to improve the pressure profile on the contacted tissue and to avoid restricting blood flow and other negative impacts. In the illustrated embodiment, the femoral paddle 705 is attached to the front straps 717 by passing under securement plates 1701 and 1703, which are mounted to the femoral paddle 705 via screws 1705. The front straps 717 pass between the paddle 705 and the securement plates 1701 and 1703 with minimal friction. The sleeves do not inhibit the movement of the front straps 717 or prevent the adjustment of hip paddle 701 relative to ischial seat paddle 703. FIGS. 17B, 17C, and 17D illustrate varying degrees of convexity according to varying embodiments of the present invention.

FIGS. 18A, 18B, and 18C illustrate one embodiment of a rear pad 725, wherein the rear pad 725 is further operable to be attached the proximal body attachment portion 700 differently than ischial seat paddle 703 and hip paddle 701. In this illustrated embodiment, rear pad 725 is slidably attached to the rear straps 715 between ischial seat paddle 703 and hip paddle 701. This construction gives the rear pad 725 freedom of movement for placement between the ischial seat region 1B (FIG. 1), and outer hip/thigh region 1C (FIG. 1). Rear pad 725 is operable to translate vertically, slide circumferentially (i.e., around perimeter of a received leg), or rotate in a vertical plane to provide continuous rather than discrete adjustability in order to tailor a more customized, comfortable, and secure fit for a user. The rear pad 725 is further constructed with a curvature that is convex with respect to the contacted region of the body, or is otherwise flat. The convex shape aids in correct ergonomic placement of the paddle to improve the pressure profile on the contacted tissue and to avoid restricting blood flow and other negative impacts. The rear pad 725 is attached to the rear straps 715 in a way that allows easy movement of the pad and provides flexibility to a user. In the illustrated embodiment, the rear pad 725 is constructed from at least a pad backing 1801, pad sleeves 1803, and pad cushion 1805. Pad sleeves 1803, of which there is at least one but preferably one for each rear strap 715, are attached to the pad backing 1801 in a manner that allows rear straps 715 to pass through with minimal friction. The sleeves do not inhibit the movement of the rear straps 715 or prevent the adjustment of hip paddle 701 relative to the ischial seat paddle 703. On an opposite side of the pad backing 1801, a pad cushion is attached to the pad backing 1801 in a manner that avoids irritation of the user. For example, the pad cushion 1805, in one embodiment, is bonded or stitched to the pad backing 1801. The rear pad is placed between the residual limb of the user and the rear pad such that residual limb tissue is not pinched between the straps 715 during usage. FIGS. 18B and 18C illustrate varying degrees of convexity according to varying embodiments of the present invention, wherein the rear pad 725 of FIG. 18B is flat and the rear pad 725 of FIG. 18C is slightly convex.

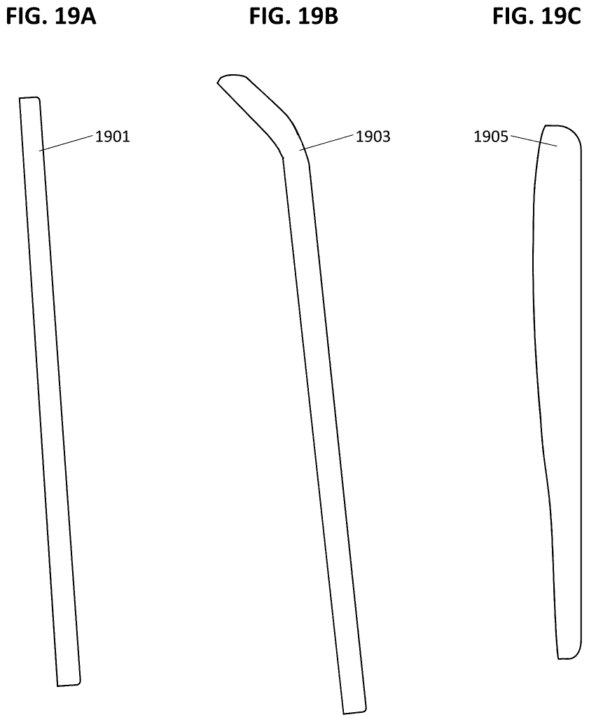

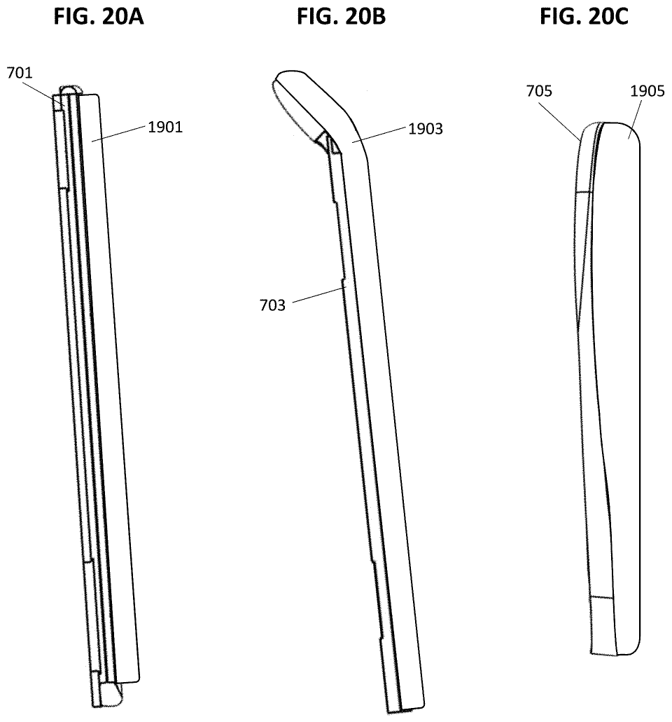

FIGS. 19A, 19B, 19C, 20A, 20B, and 20C illustrate embodiments wherein there is a layer of padding between each paddle and the user's leg. FIGS. 19A and 20A illustrate a pad on the hip paddle (701), FIGS. 19B and 20B illustrate a pad on the ischial seat paddle (703), and FIGS. 19C and 20C illustrate a pad on the femoral paddle (705). Pads (1901, 1903, and 1905) are attached to the paddles in a semi-permanent or permanent manner, for example via physical, mechanical, or chemical means. In one embodiment, the pads are bonded to the paddle with an adhesive. In another embodiment, the pads are attached by hook-and-loop tape. In one embodiment, the padding is between 1 mm (0.0394 inches) and 10 mm (0.394 inches) thick, and in a preferred embodiment is 5 mm (0.197 inches) thick.

In constructing a prosthetic, variables such as weight, strength, and durability are key factors in material selection in order to balance comfort and usability with strength and endurance. Many modern sockets today are constructed from carbon fiber reinforced thermoplastics and similar materials. However, these materials must be molded to a user's residual limb, and minor adjustments are often impractical or impossible. Advantageously, rods of the present invention are constructed in a highly adjustable manner, wherein supporting rods and paddles provide many degrees of freedom for initial or continuous fit adjustment. The rods of the present invention are, in one embodiment, constructed from basalt, including basalt fiber or a basalt fiber composite. Basalt rods provide a lightweight alternative to steel rods while providing a higher specific and tensile strength with a lower modulus of elasticity. Basalt is also naturally resistant to corrosion and rust, eliminating any potential issues caused by water and sweat. Thus, basalt ensures high durability while remaining lightweight. In contrast to traditional socket implementations, this embodiment of the present invention allows for paddles and padding to ensure the flexibility needed for comfort and usability, wherein the rods provide support without requiring as much flexibility.

In another embodiment, the rods are constructed from any other metal known in the art, including steel, aluminum, titanium, or composites thereof. Alternatively, the rods are constructed from carbon fiber, epoxy, plastic, or carbon fiber reinforced metal, epoxy, or plastic. In one embodiment, the hip paddle, ischial seat paddle, femoral paddle, the baseplate assembly, and subcomponents of each of these elements are constructed from a plastic or reinforced thermoplastic, which is preferably produced by additive manufacturing (three-dimensional (3D) printing) or injection molding. Alternatively, each of these elements are constructed from metal, metal alloys, or basalt materials. In another embodiment, the rear pad is constructed from a padding material, such as a textile, leather, or rubber, wherein a reinforcing element is constructed from plastic, reinforced thermoplastic, metal, metal alloy, or basalt.

Preferably, padding is applied to the paddles, wherein the padding is constructed from high-density, closed-cell, ethylene vinyl acetate (EVA). In another embodiment, the padding is constructed from any other polymer, plastic, or foam that provides both comfort and lightweight padding. In one embodiment, the padding is between 1 mm (0.0394 inches) and 10 mm (0.394 inches) thick, and in a preferred embodiment is approximately 5 mm (0.197 inches) thick. In another embodiment, padding is attached to the inside face of the straps in order to provide increased patient comfort. The strap padding is, in one embodiment, between 1 mm (0.0394 inches) and 7 mm (0.276 inches) thick, and in a preferred embodiment the padding is approximately 3 mm (0.118 inches) thick. The straps further include a coated back, which in one embodiment is 1/16 inch (1.588 mm) low-density polyethylene (LDPE).

This design improves upon previous designs by adding a level of adjustability. Across individuals the precise location of the hip, femur, and ischial seat may vary. The design contains this circumferential adjustability component in order to ensure the limb is properly supported, as well as a radial component to provide seamless connection of the socket to the limb; both of which work to normalize a user's posture and gait as much as possible.

In one embodiment, the present invention further includes a tissue containment unit, wherein the tissue containment unit maintains contact between a residual limb and the socket to prevent slippage and maintain comfort, specifically during the unloading phase of a user's gait. The unit keeps limb tissue within the socket and eliminates uncomfortable pinching or undistributed pressure from limb size changes throughout usage. The tissue containment unit is preferably breathable while increasing friction between a residual limb and a paddle surface. In one embodiment, the unit includes an anti-slip material on an outside surface, wherein the anti-slip material interfaces with an internal surface of a hip paddle, ischial seat paddle, and/or femoral paddle. Anti-slip materials include any of polyvinyl chloride (PVC), polyethylene (PE), hard polyethylene (HDPE), cross-linked polyethylene (XLPE), thermoplastic liquid crystal (thermochrom), Polybenzimidazole (PBI), thermoplastic polyurethane (TPU), vinyl polymer, thermoplastic elastomer (TPE), polyolefin (POE), polyisobutylene (PIB), ethylene-propylene rubber (EPR), ethylene-propylene-diene rubber (EPDM), polypropylene (PP), polybutylene (PB), natural rubber, silicone rubber, polyisoprene synthetic rubber, latex, polytetrafluoroethylene (PTFE), elastomer, thermoplastic rubber, liquid silicone rubber, polyurethane (PU), ethylvinyl acetate (EVA), phthalate, bioplastic, and/or biopolymer. One or more of these materials are integrated on top of or within the tissue containment unit. In another embodiment, the materials further include additional embedded frictional elements, such as minerals, plastic beads, or rubber pellets, which provide additional surface area and increase coefficients of friction between the unit and the paddles. These materials are, in another embodiment, constructed into shapes, thicknesses, patterns, and designs that increase friction between the unit and the paddles. For example, in one embodiment, the unit includes strips of silicone rubber arranged in wave formation. Preferably, the unit includes separated zones of frictional materials, wherein the separated zones correspond to contact areas with the paddles. For example, in one embodiment, the unit includes an ischial zone, a hip zone, a rear pad zone, and a femoral zone, and wherein these zones correspond to ischial seat paddles, hip paddles, rear pad, and femoral paddles, and wherein a remainder of the unit is absent of frictional material to maintain breathability. In one embodiment, the unit includes perforations to improve breathability. The unit further includes one or more layers of woven or non-woven base material, such as spandex, nylon, polyester, wool, cotton. In one embodiment, the unit is integrated within the socket, wherein the unit is attached to paddles, rods, straps, or other elements of the socket via any physical, mechanical, or chemical means, including hook-and-loop surfaces, adhesives, straps, snaps, buttons, latches, screws, bolts, nails, or any other attachment mechanism known in the art. For example, in one embodiment, one or more components of at least one baseplate, paddle, and/or strap includes one half of a hook-and-loop surface, and the unit includes a second half of a hook-and-loop surface that is embedded within or attached to the unit. Preferably, the unit is removable for easy washing. In another embodiment, the unit is separate from the socket, wherein the unit includes each of the frictional zones but is not attached to the socket. The unit is, in one embodiment, constructed to be used with an additional liner. In another embodiment, the unit is constructed to be used as a stand-alone liner.

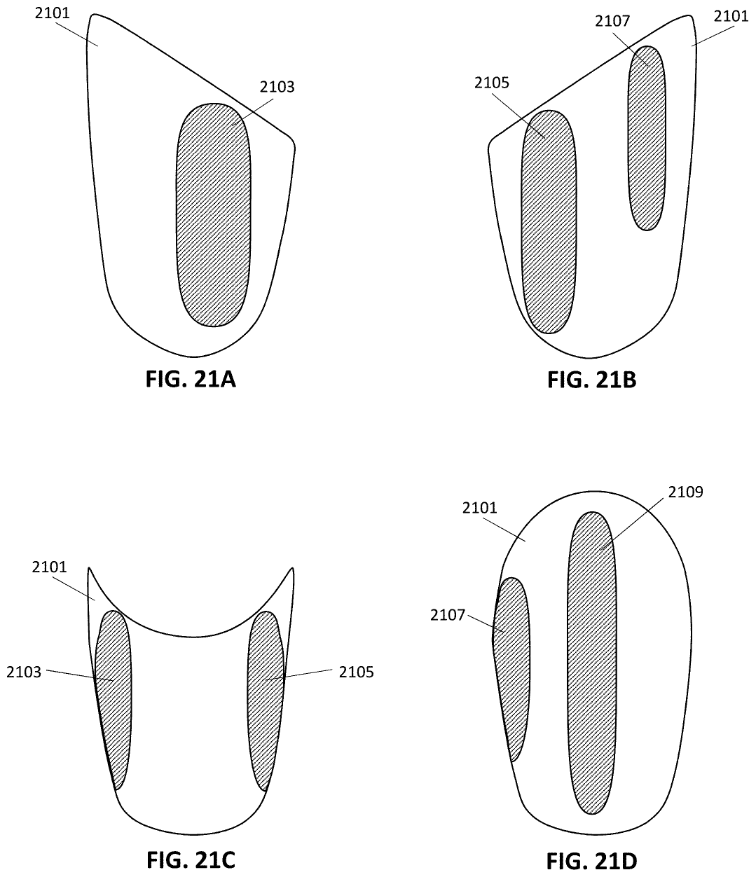

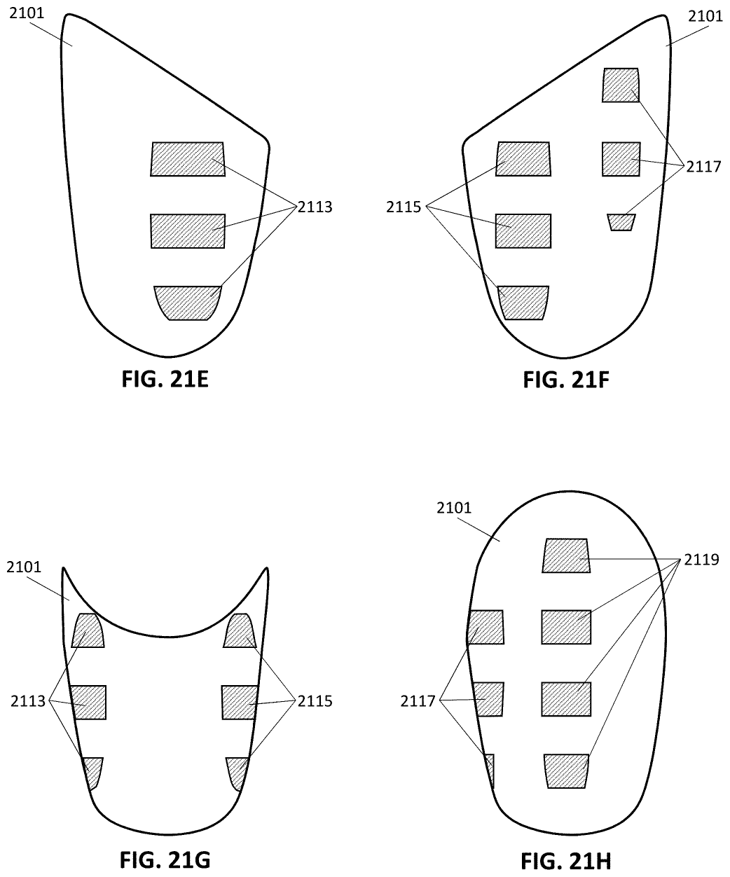

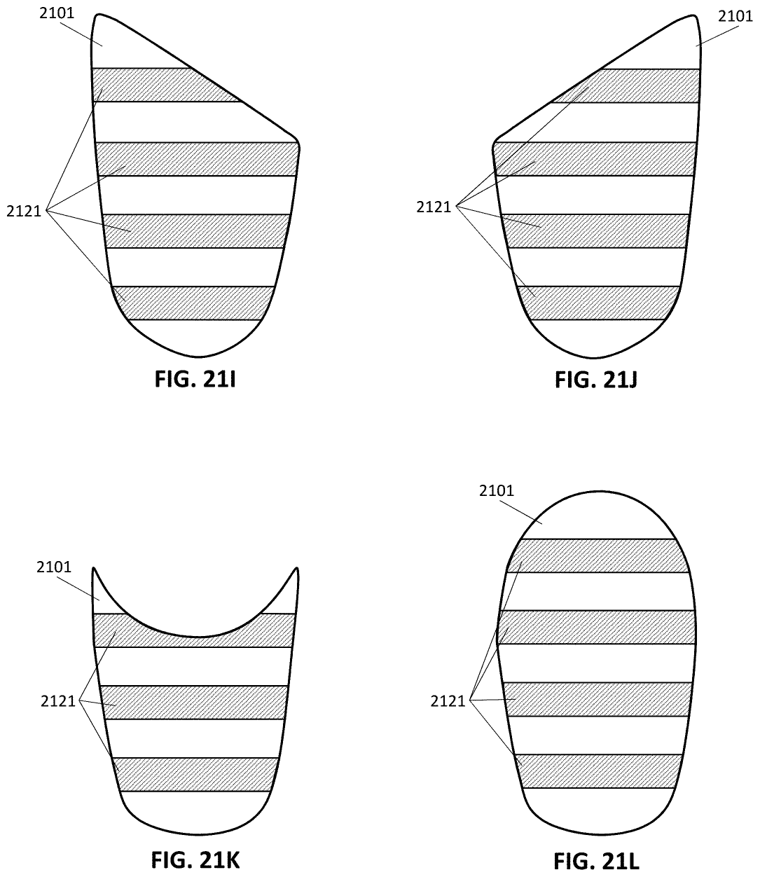

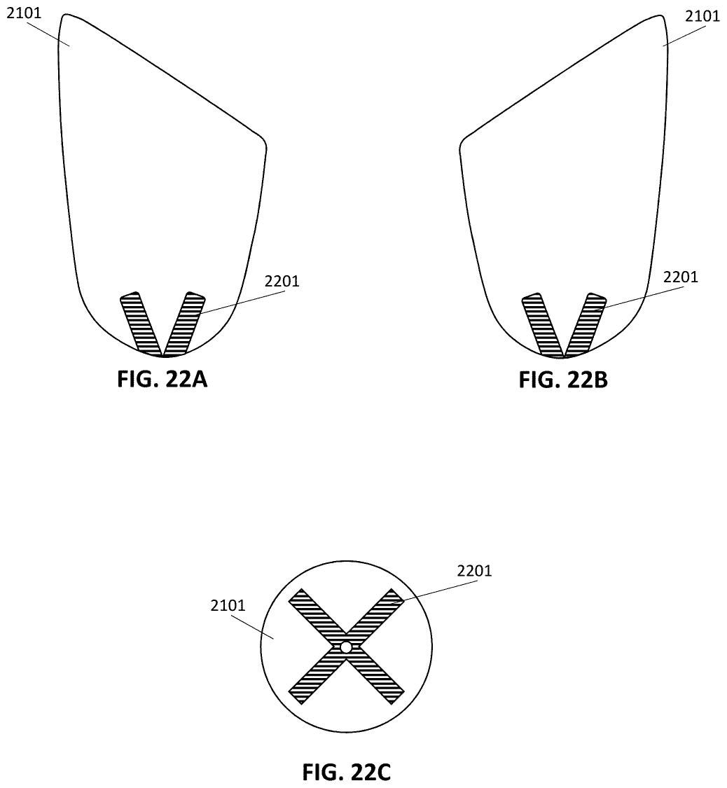

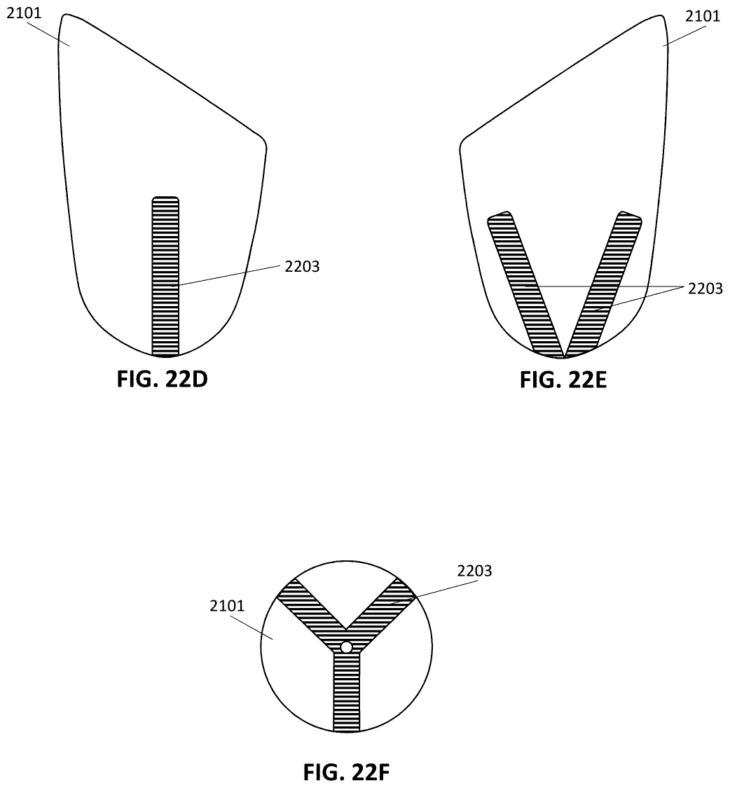

FIGS. 21A, 21B, 22C, and 22D illustrate a first embodiment of the separated zones of frictional materials. FIG. 21A illustrates a front of the tissue containment unit 2101 and a femoral zone of frictional material 2103. FIG. 21B illustrates a back of the tissue containment unit 2101 and both an ischial zone of frictional material 2105 and a rear pad zone of frictional material 2107. FIG. 21C illustrates a medial side of the tissue containment unit 2101 and both an ischial zone of frictional material 2105 and a femoral zone of frictional material 2013. FIG. 21D illustrates the lateral side of the tissue containment unit 2101 and both a rear pad zone of frictional material 2107 and hip zone of frictional material 2109.

FIGS. 21E, 21F, 21G, and 21H illustrate a second embodiment of the separated zones of frictional materials, wherein the zones of frictional materials have a smaller area and allow for increased breathability of the socket. The frictional zones are targeted to align with contact points within the socket (e.g., with paddles) FIG. 21E illustrates a front of the tissue containment unit 2101 and femoral zones of frictional material 2113. FIG. 21F illustrates a back of the tissue containment unit 2101 and both ischial zones of frictional material 2115 and rear pad zones of frictional material 2117. FIG. 21G illustrates a medial side of the tissue containment unit 2101 and both ischial zones of frictional material 2115 and femoral zones of frictional material 2113. FIG. 21H illustrates the lateral side of the tissue containment unit 2101 and both rear pad zones of frictional material 2117 and hip zones of frictional material 2119.

FIGS. 21I, 21J, 21K, and 21L illustrate a third embodiment of the separated zones of frictional materials, wherein the zones of frictional materials extend around the tissue containment unit 2101 as continuous rings of frictional material 2121. FIG. 21I illustrates a front view of the tissue containment unit 2121, FIG. 21J illustrates a back view of the tissue containment unit 2101, FIG. 21K illustrates a medial view of the tissue containment unit 2101, and FIG. 21L illustrates a lateral view of the tissue containment unit 2101, all illustrating the rings of frictional material 2101.

FIGS. 22A, 22B, and 22C illustrate one embodiment of a tissue containment unit 2101 with an X-shape according to the present invention. The tissue containment unit 2101 is configured to include improved means of tissue restriction and containment unit reinforcement. A reinforcement element 2201 is integrated into the tissue containment unit to prevent tissue from unevenly pushing out of different areas of the tissue containment unit, which would result in uneven pressure and discomfort. FIG. 22A illustrates one embodiment of the front of the tissue containment unit 2101 and a reinforcement element 2201. FIG. 22B illustrates one embodiment of the back of the tissue containment unit 2101 and a reinforcement element 2201. FIG. 22C illustrates one embodiment of a distal end of the tissue containment unit 2101 and a reinforcement element 2201. In one embodiment, the reinforcement element includes multiple spokes extending outwards from a center, most distal point of the tissue containment unit. In another embodiment, the reinforcement element 2201 is a set of concentric circles radiating outwards from the center, most distal point of the tissue containment unit. In yet another embodiment, the reinforcement element 2201 connects all of the separated zones of frictional materials. Notably, the reinforcement element 2201 is operable to be constructed with any shape known in the art, including a triangle, rectangle, star, strips, waves, or other linear or polygonal design. The reinforcement element is integrated on an inside surface, an outside surface, or within the tissue containment unit 2101. In one embodiment, the reinforcement zone is constructed with a material that is stiffer than the tissue containment unit such that it creates a rigid or semi-rigid shape, such as plastic, silicone rubber, metal, wood, or other material. Alternatively, the reinforcement element 2201 is the same material as the tissue containment unit 2101 but is a zone formed of folds, pleats, or any other method commonly used for strengthening of flexible materials. In another embodiment, the reinforcement element 2201 is constructed from any woven or non-woven, natural or synthetic fabric, including nylon, cotton, wool, silk, polyester, polyethylene, or polypropylene.

FIGS. 22D, 22E, and 22F illustrate a preferred embodiment of the reinforcement elements, previously described in detail with respect to FIGS. 22A, 22B, and 22C, wherein the reinforcement element includes a Y-shape corresponding to three paddles or to areas between the three paddles. FIG. 22D illustrates one embodiment of the front of the tissue containment unit 2101 and a reinforcement element 2203. FIG. 22E illustrates one embodiment of the back of the tissue containment unit 2101 and a reinforcement element 2203. FIG. 22F illustrates one embodiment of a distal end of the tissue containment unit 2101 and a reinforcement element 2203. FIGS. 22D, 22E, and 22F illustrate an embodiment where the reinforcement element 2203 extends to contact with paddles securing the socket to the residual limb during use, for example, the hip paddle 701, ischial seat paddle 703, and femoral paddle 705. In one embodiment, the reinforcement element 2203 is connected to the paddles. In another embodiment, the reinforcement element 2203 matingly contacts the paddles. In a further embodiment, the reinforcement element 2203 is positioned between the paddles. In yet another embodiment, the reinforcement element 2203 is attached to the front straps and/or the backstraps.

The above-mentioned examples are provided to serve the purpose of clarifying the aspects of the invention, and it will be apparent to one skilled in the art that they do not serve to limit the scope of the invention. By nature, this invention is highly adjustable, customizable and adaptable. The above-mentioned examples are just some of the many configurations that the mentioned components can take on. All modifications and improvements have been deleted herein for the sake of conciseness and readability but are properly within the scope of the present invention.

What is claimed is:

1. A socket for a prosthetic leg, comprising:
at least three paddles, including a femoral paddle, a hip paddle, and an ischial seat paddle;
a baseplate assembly; and
at least one front strap and at least one rear strap;
wherein the at least one front strap and the at least one rear strap are attached to the hip paddle and the ischial seat paddle;
wherein the femoral paddle is attached to the at least one front strap;
wherein the baseplate assembly is connected to the hip paddle via at least one rod, and wherein the baseplate assembly is connected to the ischial seat paddle via at least two rods;
wherein a height of the hip paddle is adjustable along the at least one rod, and wherein a height of the ischial seat paddle is adjustable along the at least two rods;
wherein the baseplate assembly includes an ischial seat connector, a hip rod connector, and a knee connector;
wherein the hip rod connector is adjustable in at least one first radial direction and at least one first circumferential direction;
wherein the hip rod connector is angled between 5 degrees and 10 degrees from a top surface of the baseplate assembly;
wherein the ischial seat connector is continuously, circumferentially adjustable; and
wherein the knee connector is adjustable in at least one second radial direction and at least one second circumferential direction.

2. The socket of claim 1, further comprising at least one front pad and at least one rear pad, wherein the at least one front pad is attached to the at least one front strap, and wherein the at least one rear pad is attached to the at least one rear strap.

3. The socket of claim 1, wherein the ischial seat connector is located along an outer edge of the baseplate assembly.

4. The socket of claim 1, wherein the hip connector is connected to a center of the baseplate assembly, and wherein the hip connector is extendable radially from the center of the baseplate assembly.

5. The socket of claim 1, wherein the knee connector is positioned on a bottom of the baseplate assembly, wherein the knee connector is connected to a center of the baseplate assembly, and wherein the knee connector is extendable radially from the center of the baseplate assembly and/or is adjustable circumferentially.

6. The socket of claim 1, wherein the baseplate assembly restricts radial adjustability of the hip rod connector by restricting motion of an arm of the hip rod connector via at least one slider slot.

7. The socket of claim 1, wherein the socket is reversible for left-limb and right-limb usage.

8. The socket of claim 7, wherein a baseplate of the baseplate assembly is reversible, and wherein the hip rod connector and the ischial seat connector are operable to connect to the baseplate in a mirrored position from a non-reversed orientation.

9. A socket for a prosthetic leg, comprising:
at least two paddles, including a hip paddle and an ischial seat paddle;
a baseplate assembly; and
at least one front strap and at least one rear strap;
wherein the at least one front strap and the at least one rear strap are attached to the hip paddle and the ischial seat paddle;
wherein the hip paddle and the ischial seat paddle are each connected to the baseplate assembly via at least one rod;
wherein the baseplate assembly includes an ischial seat connector and a hip rod connector;
wherein the hip rod connector is adjustable in at least one radial direction and at least one circumferential direction; and
wherein a hip paddle attachment point of a first strap of the at least one front strap is located a greater distance from the baseplate assembly than an ischial seat paddle attachment point of the first strap of the at least one front strap, such that the first strap of the at least one front strap is configured to be approximately parallel to an inguinal ligament.

10. The socket of claim 9, wherein the at least one front strap is adjustable via a ratcheting mechanism.

11. The socket of claim 9, wherein a hip paddle attachment point of a first strap of the at least one rear strap is located a greater distance from the baseplate assembly than an ischial seat paddle attachment point of the first strap of the at least one rear strap, such that the first strap of the at least one rear strap is configured to support a gluteal fold.

12. The socket of claim 9, wherein the at least one front strap comprises at least two front straps and the at least one rear strap comprises at least three rear straps.

13. The socket of claim 9, further comprising a femoral paddle, wherein the femoral paddle is attached to the at least one front strap.

14. The socket of claim 9, wherein the hip paddle and the ischial seat paddle each include at least two panel components, wherein the at least one front strap and the at least one rear strap are positioned and secured between the at least two panel components.

15. A socket for a prosthetic leg, comprising:
at least three paddles, including a femoral paddle, a hip paddle, and an ischial seat paddle;
a baseplate assembly; and
at least one front strap and at least one rear strap;
wherein the femoral paddle is attached to the at least one front strap;
wherein the at least one front strap and the at least one rear strap are attached to the hip paddle and the ischial seat paddle;
wherein the hip paddle and the ischial seat paddle are each connected to the baseplate assembly via at least one rod;
wherein the baseplate assembly includes an ischial seat connector and a hip rod connector;
wherein the ischial seat connector and the hip rod connector are attached to a top surface of the baseplate assembly;
wherein the hip rod connector is angled between 5 degrees and 10 degrees from a top surface of the baseplate assembly; and
wherein the hip rod connector is adjustable in at least one radial direction and at least one circumferential direction.

16. The socket of claim 15, wherein the socket is reversible.

17. The socket of claim 15, wherein the hip rod connector governs an internal volume the socket is operable to contain.

18. The socket of claim 15, wherein a height of the hip paddle is adjustable along a first rod of the at least one rod, and wherein a height of the ischial seat paddle is adjustable along a second rod of the at least one rod.

19. The socket of claim 15, further comprising at least one tissue containment unit, wherein the tissue containment unit includes frictional zones corresponding to contact points with the at least three paddles and at least one pad.

20. The socket of claim 19, wherein the at least one tissue containment unit is operable to be attached to at least one internal surface of the socket.
